(19) United States
(12) Patent Application Publication
(10) Pub. No.: US 2010/0221800 A1
(43) Pub. Date: Sep. 2, 2010

(54) MICROORGANISM ENGINEERED TO PRODUCE ISOPROPANOL

(75) Inventors: James C. Liao, Los Angeles, CA (US); Shota Atsumi, Davis, CA (US); Taizo Hanai, Fukuoka (JP)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, Oakland, CA (US)

(21) Appl. No.: 12/682,554

(22) PCT Filed: Oct. 11, 2008
(86) PCT No.: PCT/US08/79667

§ 371 Date: Apr. 9, 2010

Related U.S. Application Data
(60) Provisional application No. 60/979,731, filed on Oct. 12, 2007.

(51) Int. Cl. C12P 7/04; C12N 1/00; C12N 1/21; C12N 15/74; C12N 15/63

(52) U.S. Cl. 435/157; 435/243; 435/252.33; 435/252.3; 435/471; 435/320.1

(57) ABSTRACT

Provided herein are metabolically-modified microorganisms useful for producing biofuels.

### CROSS REFERENCE TO RELATED APPLICATIONS

This application claims priority to U.S. Provisional Application Ser. No. 60/979,731, filed Oct. 12, 2007, the disclosure of which is incorporated herein by reference.

### TECHNICAL FIELD

Metabolically-modified microorganisms and methods of producing such organisms are provided. Also provided are methods of producing biofuels by contacting a suitable substrate with a metabolically-modified microorganism and enzymatic preparations there from.

### BACKGROUND

Demand for biofuels as a substitute for petroleum is expected to increase because of economic and environmental concerns. The common bio-fuel, ethanol, is not ideal because it has a lower energy density than gasoline and must be mixed with gasoline at a limited concentration range in order to serve as a transportation fuel. Ethanol is also hygroscopic and corrosive, which poses a problem for storage and distribution systems. Isopropanol has the same advantages as 1-butanol over ethanol; with the added advantage that isopropanol has a higher octane number than 1-butanol because of its branched carbon chain. 1-Butanol has been produced as a fermentation product and used as a motor fuel, but isopropanol has never been produced from a renewable source in high yield and has not been considered as a gasoline substitute, even though it has been used as an engine additive.

### SUMMARY

Provided herein are metabolically-modified microorganisms that include recombinant biochemical pathways useful for producing biofuels such as isopropanol. Also provided are methods of producing biofuels using microorganisms described herein.

The disclosure provides a recombinant microorganism comprising a biochemical pathway to produce isopropanol from fermentation of a suitable carbon substrate the biochemical pathway comprising an acetyl-CoA acetyltransferase, wherein the microorganism comprises at least one heterologous polypeptide compared to a corresponding parental microorganism. In one embodiment, the microorganism comprises elevated expression of a polypeptide having acetyl-CoA acetyltranferase activity, as compared to a parental microorganism, wherein the recombinant microorganism produces a metabolite comprising acetoacetyl-CoA from a substrate comprising acetyl-CoA. In one embodiment, the polypeptide having acetyl-CoA acetyltranferase activity is encoded by an atoB gene or homolog thereof, or a fadA gene or homolog thereof. In another embodiment, the polypeptide having acetyl-CoA acetyltranferase activity is encoded by a polynucleotide having at least about 50% identity to a sequence as set forth in SEQ ID NO:1. The atoB gene or fadA gene can be derived from the genus Escherichia. In one embodiment, the microorganism is a recombinant E. coli. In yet a further embodiment, the polypeptide having acetyl-CoA acetyltransferase activity is encoded by a thl gene or homolog thereof. In one embodiment, the thl gene is derived from the genus Clostridium. In yet another embodiment, the microorganism comprises elevated expression of a polypeptide having acetoacetyl-CoA transferase activity, as compared to a parental microorganism, wherein the recombinant microorganism produces a metabolite comprising acetoacetate from a substrate comprising acetoacetyl-CoA. In a further embodiment, the acetoacetyl-CoA transferase is encoded by an atoAD gene or homolog thereof (e.g., atoD comprises a sequence set forth in SEQ ID NO:3). In one embodiment, the atoD comprises a sequence that is at least 50%, 60% or 70% or more identical to SEQ ID NO:3. In another embodiment, the atoAD is derived from E. coli. In yet another embodiment, the microorganism comprises an elevated expression of a polypeptide having acetoacetate decarboxylase activity, as compared to a parental microorganism, wherein the recombinant microorganism produces a metabolite comprising acetone from a substrate comprising acetoacetate. In another embodiment, the microorganism comprises an acetoacetate decarboxylase encoded by an adc gene or homolog thereof. In one embodiment, the adc gene is derived from Clostridium acetobutylicum, Butyrivibrio fibrisolvens, Thermoanaerobacterium thermosaccharolyticum, and Clostridium difficile. In a specific embodiment, the microorganism is Clostridium acetobutylicum. In yet another embodiment, the acetoacetate decarboxylase comprises a sequence that is at least 50%, 60%, 70%, 80%, 90%, 95%, 995 identical to SEQ ID NO:5. In another embodiment, the microorganism comprises elevated expression of a polypeptide having secondary alcohol dehydrogenase activity, as compared to a parental microorganism, wherein the recombinant microorganism produces a metabolite comprising isopropanol from a substrate comprising acetone. In one embodiment, the polypeptide having secondary alcohol dehydrogenase activity is encoded by an adh or sadh gene or homolog thereof. In yet another embodiment, the adh gene is derived from T. brockii or C. beijerinckii. In yet a further embodiment, the adh or sadh comprises a sequence that is at least 50%, 60%, 70%, 80%, 90%, 95%, 995 identical to SEQ ID NO:7 or 9 and has alcohol dehydrogenase activity. In one embodiment, the microorganism comprises an expression or increased expression of an acetyl-CoA acetyltransferase, an acetoacetyl-CoA transferase, an acetoacetate decarboxylase and an adh (secondary alcohol dehydrogenase).

The disclosure also provides a recombinant microorganism comprising a recombinant biochemical pathway to produce isopropanol from fermentation of a suitable carbon substrate, wherein the recombinant biochemical pathway comprises elevated expression of an acetyl-CoA acetyltransferase, an acetoacetyl-CoA transferase, an acetoacetate decarboxylase and an adh (secondary alcohol dehydrogenase).

The disclosure further provides a method of producing a recombinant microorganism that converts a suitable carbon substrate to isopropanol, the method comprising transforming a microorganism with one or more polynucleotides encoding polypeptides having an acetyl-CoA acetyltransferase, an acetoacetyl-CoA transferase, an acetoacetate decarboxylase and an adh (secondary alcohol dehydrogenase) activity.

The disclosure provides a method for producing isopropanol, the method comprising inducing over-expression of an thl or atoB gene, a ctfAB or atoAD gene or operon, an adc gene, an adh (secondary alcohol dehydrogenase) gene, or any combination thereof, in an organism, wherein the organism produces isopropanol when cultured in the presence of a suitable carbon substrate.

The disclosure provides a method of producing isopropanol comprising culturing a microorganism above, under conditions whereby isopropanol is produced.

The disclosure also provides a method for producing isopropanol, the method comprising: (i) inducing over-expression of a thl or atoB gene in an organism; (ii) inducing over-expression of an of a ctfAB or atoAD gene in an organism; (iii) inducing over-expression of a adc gene in the organism; (iv) inducing over-expression of an adh gene in the organism; or (v) inducing over-expression of (i), (ii), (iii), and (iv).

The disclosure provides a recombinant vector comprising: (i) a first polynucleotide encoding a first polypeptide that catalyzes the conversion of acetoacetate from a substrate comprising acetoacetyl-CoA; (ii) a second polynucleotide encoding a second polypeptide the catalyzes the conversion of acetone from a substrate comprising acetoacetate; and (iii) a third polynucleotide encoding a third polypeptide that catalyzes the conversion of isopropanol from a substrate comprising acetone. In one embodiment, the vector can be a plasmid. In another embodiment, the vector can be an expression vector. Furthermore, the vector can be used to transform or transfect a host cell (e.g., a microorganism). The transfected or transformed microorganism can be used to produce isopropanol.

The disclosure provides a recombinant microorganism comprising at least one heterologous nucleic acid sequence that facilitates the conversion of glucose to isopropanol.

The details of one or more embodiments of the disclosure are set forth in the accompanying drawings and the description below. Other features, objects, and advantages will be apparent from the description and drawings, and from the claims.

### BRIEF DESCRIPTION OF THE FIGURES

FIG. 1 shows a metabolic pathway for isopropanol production.

FIG. 2 shows a comparison of maximum isopropanol production by each combination of pathway genes.

FIG. 3 shows a time course of isopropanol production by TA11 (pTA39/pTA36: thl-atoAD-adc-adh (cb)).

FIG. 4 shows sequences of adh polynucleotides (SEQ ID NO: 7 and 9) useful in the methods and compositions of the disclosure.

### DETAILED DESCRIPTION

Unless specifically noted otherwise herein, the definitions of the terms used are standard definitions used in the art of pharmaceutical sciences. As used in the specification and the appended claims, the singular forms “a,” “an” and “the” include plural referents unless the context clearly dictates otherwise. Thus, for example, reference to “a substrate” includes mixtures of two or more such substrates, and the like.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the disclosure. Nothing herein is to be construed as an admission that the inventors are not entitled to antedate such disclosure by virtue of prior disclosure.

The native producers of 1-butanol, such as Clostridium acetobutylicum, also produce byproducts such as acetone, ethanol, and butyrate as fermentation products. High 1-butanol producers have been developed to optimize the yield and productivity. However, most of these microorganisms are relatively difficult to manipulate. Genetic tools for these organisms are not as efficient as those for user-friendly hosts such as E. coli and physiology and their metabolic regulation are much less understood, prohibiting rapid progress towards high-efficiency production. Furthermore, no native microorganisms have been identified to produce from glucose to other higher alcohols such as isopropanol, 2-methyl 1-butanol, 3-methyl 1-butanol, and 2-phenylethanol to industrially relevant quantities, despite the small amounts that have been identified as microbial byproducts.

In various embodiments the metabolically engineered microorganisms provided herein include biochemical pathways for the production of alcohols including isopropanol. In various aspects a recombinant microorganism provided herein includes the elevated expression of at least one target enzyme as compared to a parental microorganism. The target enzyme is encoded by, and expressed from, a nucleic acid sequence derived from a suitable biological source. In some aspects the nucleic acid sequence is a gene derived from a bacterial or yeast source.

As used herein, the term “metabolically engineered” or “metabolic engineering” involves rational pathway design and assembly of biosynthetic genes, genes associated with operons, and control elements of such nucleic acid sequences, for the production of a desired metabolite, such as an alcohol, in a microorganism. “Metabolically engineered” can further include optimization of metabolic flux by regulation and optimization of transcription, translation, protein stability and protein functionality using genetic engineering and appropriate culture condition. The biosynthetic genes can be heterologous to the host (e.g., microorganism), either by virtue of being foreign to the host, or being modified by mutagenesis, recombination, and/or association with a heterologous expression control sequence in an endogenous host cell. Appropriate culture conditions are conditions of culture medium pH, ionic strength, nutritive content, etc.; temperature; oxygen/CO₂/nitrogen content; humidity; and other culture conditions that permit production of the compound by the host microorganism, i.e., by the metabolic action of the microorganism. Appropriate culture conditions are well known for microorganisms that can serve as host cells.

Accordingly, metabolically “engineered” or “modified” microorganisms are produced via the introduction of genetic material into a host or parental microorganism of choice thereby modifying or altering the cellular physiology and biochemistry of the microorganism. Through the introduction of genetic material the parental microorganism acquires new properties, e.g. the ability to produce a new, or greater quantities of, an intracellular metabolite. In an illustrative embodiment, the introduction of genetic material into a parental microorganism results in a new or modified ability to produce an alcohol such as isopropanol. The genetic material introduced into the parental microorganism contains gene(s), or parts of genes, coding for one or more of the enzymes involved in a biosynthetic pathway for the production of an alcohol and may also include additional elements for the expression and/or regulation of expression of these genes, e.g. promoter sequences.

Microorganisms provided herein are modified to produce metabolites in quantities not available in the parental microorganism. A “metabolite” refers to any substance produced by metabolism or a substance necessary for or taking part in a particular metabolic process. A metabolite can be an organic compound that is a starting material (e.g., glucose or pyruvate) in, an intermediate (e.g., Acetyl-CoA) in, or an end product (e.g., isopropanol) of metabolism. Metabolites can be used to construct more complex molecules, or they can be broken down into simpler ones. Intermediate metabolites may be synthesized from other metabolites, perhaps used to make more complex substances, or broken down into simpler compounds, often with the release of chemical energy. End products of metabolism are the final result of the breakdown of other metabolites.

The term “biosynthetic pathway”, also referred to as “metabolic pathway”, refers to a set of anabolic or catabolic biochemical reactions for converting (transmuting) one chemical species into another. Gene products belong to the same “metabolic pathway” if they, in parallel or in series, act on the same substrate, produce the same product, or act on or produce a metabolic intermediate (i.e., metabolite) between the same substrate and metabolite end product.

The term “substrate” or “suitable substrate” refers to any substance or compound that is converted or meant to be converted into another compound by the action of an enzyme. The term includes not only a single compound, but also combinations of compounds, such as solutions, mixtures and other materials which contain at least one substrate, or derivatives thereof. Further, the term “substrate” encompasses not only compounds that provide a carbon source suitable for use as a starting material, such as any biomass derived sugar, but also intermediate and end product metabolites used in a pathway associated with a metabolically engineered microorganism as described herein. A “biomass derived sugar” includes, but is not limited to, molecules such as glucose, mannose, xylose, and arabinose. The term biomass derived sugar encompasses suitable carbon substrates ordinarily used by microorganisms, such as 6 carbon sugars, including but not limited to glucose, lactose, sorbose, fructose, idose, galactose and mannose all in either D or L form, or a combination of 6 carbon sugars, such as glucose and fructose, and/or 6 carbon sugar acids including, but not limited to, 2-keto-L-gulonic acid, idonic acid (IA), gluconic acid (GA), 6-phosphogluconate, 2-keto-D-gluconic acid (2 KDG), 5-keto-D-gluconic acid, 2-ketogluconatephosphate, 2,5-diketo-L-gulonic acid, 2,3-L-diketogulonic acid, dehydroascorbic acid, erythorbic acid (EA) and D-mannonic acid.

Recombinant microorganisms provided herein can express a plurality of target enzymes involved in pathways for the production of isopropanol, from using a suitable carbon substrate.

A range of microorganisms can be modified to include a recombinant metabolic pathway suitable for the production of isopropanol. Various microorganisms can act as “sources” for genetic material encoding target enzymes suitable for use in a recombinant microorganism provided herein. The term “microorganism” includes prokaryotic and eukaryotic microbial species from the Domains Archaea, Bacteria and Eucarya, the latter including yeast and filamentous fungi, protozoa, algae, or higher Protista. The terms “microbial cells” and “microbes” are used interchangeably with the term microorganism.

The term “prokaryotes” is art recognized and refers to cells which contain no nucleus or other cell organelles. The prokaryotes are generally classified in one of two domains, the Bacteria and the Archaea. The definitive difference between organisms of the Archaea and Bacteria domains is based on fundamental differences in the nucleotide base sequence in the 16S ribosomal RNA.

The term “Archaea” refers to a categorization of organisms of the division Mendosicutes, typically found in unusual environments and distinguished from the rest of the procaryotes by several criteria, including the number of ribosomal proteins and the lack of muramic acid in cell walls. On the basis of ssrRNA analysis, the Archaea consist of two phylogenetically-distinct groups: Crenarchaeota and Euryarchaeota. On the basis of their physiology, the Archaea can be organized into three types: methanogens (prokaryotes that produce methane); extreme halophiles (prokaryotes that live at very high concentrations of salt ([NaCl]); and extreme (hyper) thermophilus (prokaryotes that live at very high temperatures). Besides the unifying archaeal features that distinguish them from Bacteria (i.e., no murein in cell wall, ester-linked membrane lipids, etc.), these prokaryotes exhibit unique structural or biochemical attributes which adapt them to their particular habitats. The Crenarchaeota consists mainly of hyperthermophilic sulfur-dependent prokaryotes and the Euryarchaeota contains the methanogens and extreme halophiles.

“Bacteria”, or “eubacteria”, refers to a domain of prokaryotic organisms. Bacteria include at least 11 distinct groups as follows: (1) Gram-positive (gram+) bacteria, of which there are two major subdivisions: (1) high G+C group (Actinomycetes, Mycobacteria, Micrococcus, others) (2) low G+C group (Bacillus, Clostridia, Lactobacillus, Staphylococci, Streptococci, Mycoplasmas); (2) Proteobacteria, e.g., Purple photosynthetic+non-photosynthetic Gram-negative bacteria (includes most “common” Gram-negative bacteria); (3) Cyanobacteria, e.g., oxygenic phototrophs; (4) Spirochetes and related species; (5) Planctomyces; (6) Bacteroides, Flavobacteria; (7) Chlamydia; (8) Green sulfur bacteria; (9) Green non-sulfur bacteria (also anaerobic phototrophs); (10) Radioresistant micrococci and relatives; (11) Thermotoga and Thermosipho thermophiles.

“Gram-negative bacteria” include cocci, nonenteric rods, and enteric rods. The genera of Gram-negative bacteria include, for example, Neisseria, Spirillum, Pasteurella, Brucella, Yersinia, Francisella, Haemophilus, Bordetella, Escherichia, Salmonella, Shigella, Klebsiella, Proteus, Vibrio, Pseudomonas, Bacteroides, Acetobacter, Aerobacter, Agrobacterium, Azotobacter, Spirilla, Serratia, Vibrio, Rhizobium, Chlamydia, Rickettsia, Treponema, and Fusobacterium.

“Gram positive bacteria” include cocci, nonsporulating rods, and sporulating rods. The genera of gram positive bacteria include, for example, Actinomyces, Bacillus, Clostridium, Corynebacterium, Erysipelothrix, Lactobacillus, Listeria, Mycobacterium, Myxococcus, Nocardia, Staphylococcus, Streptococcus, and Streptomyces.

The term “recombinant microorganism” and “recombinant host cell” are used interchangeably herein and refer to microorganisms that have been genetically modified to express or over-express endogenous nucleic acid sequences, or to express non-endogenous sequences, such as those included in a vector. The nucleic acid sequence generally encodes a target enzyme involved in a metabolic pathway for producing a desired metabolite as described above. Accordingly, recombinant microorganisms described herein have been genetically engineered to express or over-express target enzymes not previously expressed or over-expressed by a parental microorganism. It is understood that the terms “recombinant microorganism” and “recombinant host cell” refer not only to the particular recombinant microorganism but to the progeny or potential progeny of such a microorganism.

A “parental microorganism” refers to a cell used to generate a recombinant microorganism. The term “parental microorganism” describes a cell that occurs in nature, i.e. a “wild-type” cell that has not been genetically modified. The term “parental microorganism” also describes a cell that has been genetically modified but which does not express or over-express a target enzyme e.g., an enzyme involved in the biosynthetic pathway for the production of a desired metabolite such as isopropanol. For example, a wild-type microorganism can be genetically modified to express or over express a first target enzyme such as thiolase. This microorganism can act as a parental microorganism in the generation of a microorganism modified to express or over-express a second target enzyme. In turn, the microorganism modified to express or over express e.g., thiolase and a second enzyme, can be modified to express or over express a third target enzyme etc. Accordingly, a parental microorganism functions as a reference cell for successive genetic modification events. Each modification event can be accomplished by introducing a nucleic acid molecule in to the reference cell. The introduction facilitates the expression or over-expression of a target enzyme. It is understood that the term “facilitates” encompasses the activation of endogenous nucleic acid sequences encoding a target enzyme through genetic modification of e.g., a promoter sequence in a parental microorganism. It is further understood that the term “facilitates” encompasses the introduction of exogenous nucleic acid sequences encoding a target enzyme in to a parental microorganism.

In another embodiment a method of producing a recombinant microorganism that converts a suitable carbon substrate to isopropanol is provided. The method includes transforming a microorganism with one or more recombinant nucleic acid sequences encoding polypeptide(s) that have a desired enzymatic function in conversion of the carbon source to the end product. Nucleic acid sequences that encode enzymes useful for generating metabolites including homologs, variants, fragments, related fusion proteins, or functional equivalents thereof, are used in recombinant nucleic acid molecules that direct the expression of such polypeptides in appropriate host cells, such as bacterial or yeast cells. It is understood that the addition of sequences which do not alter the encoded activity of a nucleic acid molecule, such as the addition of a non-functional or non-coding sequence, is a conservative variation of the basic nucleic acid. The “activity” of an enzyme is a measure of its ability to catalyze a reaction resulting in a metabolite, i.e., to “function”, and may be expressed as the rate at which the metabolite of the reaction is produced. For example, enzyme activity can be represented as the amount of metabolite produced per unit of time or per unit of enzyme (e.g., concentration or weight), or in terms of affinity or dissociation constants.

A “protein” or “polypeptide”, which terms are used interchangeably herein, comprises one or more chains of chemical building blocks called amino acids that are linked together by chemical bonds called peptide bonds. An “enzyme” means any substance, composed wholly or largely of protein, that catalyzes or promotes, more or less specifically, one or more chemical or biochemical reactions. The term “enzyme” can also refer to a catalytic polynucleotide (e.g., RNA or DNA). A “native” or “wild-type” protein, enzyme, polynucleotide, gene, or cell, means a protein, enzyme, polynucleotide, gene, or cell that occurs in nature.

A protein has “homology” or is “homologous” to a second protein if the nucleic acid sequence that encodes the protein has a similar sequence to the nucleic acid sequence that encodes the second protein. Alternatively, a protein has homology to a second protein if the two proteins have “similar” amino acid sequences. (Thus, the term “homologous proteins” is defined to mean that the two proteins have similar amino acid sequences).

As used herein, two proteins (or a region of the proteins) are substantially homologous when the amino acid sequences have at least about 60%, 65%, 70%, 75%, 80%, 85%, 90%, 91%, 92%, 93%, 94%, 95%, 96%, 97%, 98%, or 99% identity. To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). In one embodiment, the length of a reference sequence aligned for comparison purposes is at least 30%, typically at least 40%, more typically at least 50%, even more typically at least 60%, and even more typically at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

When “homologous” is used in reference to proteins or peptides, it is recognized that residue positions that are not identical often differ by conservative amino acid substitutions. A “conservative amino acid substitution” is one in which an amino acid residue is substituted by another amino acid residue having a side chain (R group) with similar chemical properties (e.g., charge or hydrophobicity). In general, a conservative amino acid substitution will not substantially change the functional properties of a protein. In cases where two or more amino acid sequences differ from each other by conservative substitutions, the percent sequence identity or degree of homology may be adjusted upwards to correct for the conservative nature of the substitution. Means for making this adjustment are well known to those of skill in the art (see, e.g., Pearson et al., 1994, hereby incorporated herein by reference).

The following six groups each contain amino acids that are conservative substitutions for one another: 1) Serine (S), Threonine (T); 2) Aspartic Acid (D), Glutamic Acid (E); 3) Asparagine (N), Glutamine (Q); 4) Arginine (R), Lysine (K); 5) Isoleucine (I), Leucine (L), Methionine (M), Alanine (A), Valine (V), and 6) Phenylalanine (F), Tyrosine (Y), Tryptophan (W).

Sequence homology for polypeptides, which is also referred to as percent sequence identity, is typically measured using sequence analysis software. See, e.g., the Sequence Analysis Software Package of the Genetics Computer Group (GCG), University of Wisconsin Biotechnology Center, 910 University Avenue, Madison, Wis. 53705. Protein analysis software matches similar sequences using measure of homology assigned to various substitutions, deletions and other modifications, including conservative amino acid substitutions. For instance, GCG contains programs such as “Gap” and “Bestfit” which can be used with default parameters to determine sequence homology or sequence identity between closely related polypeptides, such as homologous polypeptides from different species of organisms or between a wild type protein and a mutein thereof. See, e.g., GCG Version 6.1.

A typical algorithm when comparing a inhibitory molecule sequence to a database containing a large number of sequences from different organisms is the computer program BLAST (Altschul, 1990; Gish, 1993; Madden, 1996; Altschul, 1997; Zhang, 1997), especially blastp or tblastn (Altschul, 1997). Typical parameters for BLASTp are: Expectation value: 10 (default); Filter: seg (default); Cost to open a gap: 11 (default); Cost to extend a gap: 1 (default); Max. alignments: 100 (default); Word size: 11 (default); No. of descriptions: 100 (default); Penalty Matrix: BLOWSUM62.

When searching a database containing sequences from a large number of different organisms, it is typical to compare amino acid sequences. Database searching using amino acid sequences can be measured by algorithms other than blastp known in the art. For instance, polypeptide sequences can be compared using FASTA, a program in GCG Version 6.1. FASTA provides alignments and percent sequence identity of the regions of the best overlap between the query and search sequences (Pearson, 1990, hereby incorporated herein by reference). For example, percent sequence identity between amino acid sequences can be determined using FASTA with its default parameters (a word size of 2 and the PAM250 scoring matrix), as provided in GCG Version 6.1, hereby incorporated herein by reference.

It is understood that the nucleic acid sequences described above include “genes” and that the nucleic acid molecules described above include “vectors” or “plasmids.” For example, a nucleic acid sequence encoding a keto thiolase can be encoded by an atoB gene or homolog thereof, or an fadA gene or homolog thereof. Accordingly, the term “gene”, also called a “structural gene” refers to a nucleic acid sequence that codes for a particular sequence of amino acids, which comprise all or part of one or more proteins or enzymes, and may include regulatory (non-transcribed) DNA sequences, such as promoter sequences, which determine for example the conditions under which the gene is expressed. The transcribed region of the gene may include untranslated regions, including introns, 5′-untranslated region (UTR), and 3′-UTR, as well as the coding sequence. The term “nucleic acid” or “recombinant nucleic acid” refers to polynucleotides such as deoxyribonucleic acid (DNA), and, where appropriate, ribonucleic acid (RNA). The term “expression” with respect to a gene sequence refers to transcription of the gene and, as appropriate, translation of the resulting mRNA transcript to a protein. Thus, as will be clear from the context, expression of a protein results from transcription and translation of the open reading frame sequence.

The term “operon” refers two or more genes which are transcribed as a single transcriptional unit from a common promoter. In some embodiments, the genes comprising the operon are contiguous genes. It is understood that transcription of an entire operon can be modified (i.e., increased, decreased, or eliminated) by modifying the common promoter. Alternatively, any gene or combination of genes in an operon can be modified to alter the function or activity of the encoded polypeptide. The modification can result in an increase in the activity of the encoded polypeptide. Further, the modification can impart new activities on the encoded polypeptide. Exemplary new activities include the use of alternative substrates and/or the ability to function in alternative environmental conditions.

A “vector” is any means by which a nucleic acid can be propagated and/or transferred between organisms, cells, or cellular components. Vectors include viruses, bacteriophage, pro-viruses, plasmids, phagemids, transposons, and artificial chromosomes such as YACs (yeast artificial chromosomes), BACs (bacterial artificial chromosomes), and PLACs (plant artificial chromosomes), and the like, that are “episomes,” that is, that replicate autonomously or can integrate into a chromosome of a host cell. A vector can also be a naked RNA polynucleotide, a naked DNA polynucleotide, a polynucleotide composed of both DNA and RNA within the same strand, a poly-lysine-conjugated DNA or RNA, a peptide-conjugated DNA or RNA, a liposome-conjugated DNA, or the like, that are not episomal in nature, or it can be an organism which comprises one or more of the above polynucleotide constructs such as an agrobacterium or a bacterium.

“Transformation” refers to the process by which a vector is introduced into a host cell. Transformation (or transduction, or transfection), can be achieved by any one of a number of means including electroporation, microinjection, biolistics (or particle bombardment-mediated delivery), or agrobacterium mediated transformation.

Those of skill in the art will recognize that, due to the degenerate nature of the genetic code, a variety of DNA compounds differing in their nucleotide sequences can be used to encode a given amino acid sequence of the disclosure. The native DNA sequence encoding the biosynthetic enzymes described above are referenced herein merely to illustrate an embodiment of the disclosure, and the disclosure includes DNA compounds of any sequence that encode the amino acid sequences of the polypeptides and proteins of the enzymes utilized in the methods of the disclosure. In similar fashion, a polypeptide can typically tolerate one or more amino acid substitutions, deletions, and insertions in its amino acid sequence without loss or significant loss of a desired activity. The disclosure includes such polypeptides with alternate amino acid sequences, and the amino acid sequences encoded by the DNA sequences shown herein merely illustrate embodiments of the disclosure.

The disclosure provides nucleic acid molecules in the form of recombinant DNA expression vectors or plasmids, as described in more detail below, that encode one or more target enzymes. Generally, such vectors can either replicate in the cytoplasm of the host microorganism or integrate into the chromosomal DNA of the host microorganism. In either case, the vector can be a stable vector (i.e., the vector remains present over many cell divisions, even if only with selective pressure) or a transient vector (i.e., the vector is gradually lost by host microorganisms with increasing numbers of cell divisions). The disclosure provides DNA molecules in isolated (i.e., not pure, but existing in a preparation in an abundance and/or concentration not found in nature) and purified (i.e., substantially free of contaminating materials or substantially free of materials with which the corresponding DNA would be found in nature) forms.

Included within the scope of the disclosure are recombinant expression vectors that include such nucleic acids. The term expression vector refers to a nucleic acid that can be introduced into a host microorganism or cell-free transcription and translation system. An expression vector can be maintained permanently or transiently in a microorganism, whether as part of the chromosomal or other DNA in the microorganism or in any cellular compartment, such as a replicating vector in the cytoplasm. An expression vector also comprises a promoter that drives expression of an RNA, which typically is translated into a polypeptide in the microorganism or cell extract. For efficient translation of RNA into protein, the expression vector also typically contains a ribosome-binding site sequence positioned upstream of the start codon of the coding sequence of the gene to be expressed. Other elements, such as enhancers, secretion signal sequences, transcription termination sequences, and one or more marker genes by which host microorganisms containing the vector can be identified and/or selected, may also be present in an expression vector. Selectable markers, i.e., genes that confer antibiotic resistance or sensitivity, are used and confer a selectable phenotype on transformed cells when the cells are grown in an appropriate selective medium.

The various components of an expression vector can vary widely, depending on the intended use of the vector and the host cell(s) in which the vector is intended to replicate or drive expression. Expression vector components suitable for the expression of genes and maintenance of vectors in E. coli, yeast, Streptomyces, and other commonly used cells are widely known and commercially available. For example, suitable promoters for inclusion in the expression vectors of the disclosure include those that function in eukaryotic or prokaryotic host microorganisms. Promoters can comprise regulatory sequences that allow for regulation of expression relative to the growth of the host microorganism or that cause the expression of a gene to be turned on or off in response to a chemical or physical stimulus. For E. coli and certain other bacterial host cells, promoters derived from genes for biosynthetic enzymes, antibiotic-resistance conferring enzymes, and phage proteins can be used and include, for example, the galactose, lactose (lac), maltose, tryptophan (trp), beta-lactamase (bla), bacteriophage lambda PL, and T5 promoters. In addition, synthetic promoters, such as the tac promoter (U.S. Pat. No. 4,551,433), can also be used. For E. coli expression vectors, it is useful to include an E. coli origin of replication, such as from pUC, p1P, p1, and pBR.

Thus, recombinant expression vectors contain at least one expression system, which, in turn, is composed of at least a portion of PKS and/or other biosynthetic gene coding sequences operably linked to a promoter and optionally termination sequences that operate to effect expression of the coding sequence in compatible host cells. The host cells are modified by transformation with the recombinant DNA expression vectors of the disclosure to contain the expression system sequences either as extrachromosomal elements or integrated into the chromosome.

As previously noted, the term “host cell” is used interchangeably with the term “recombinant microorganism” and includes any cell type which is suitable for producing isopropanol and susceptible to transformation with a nucleic acid construct such as a vector or plasmid.

As will be understood by those of skill in the art, it can be advantageous to modify a coding sequence to enhance its expression in a particular host. The genetic code is redundant with 64 possible codons, but most organisms typically use a subset of these codons. The codons that are utilized most often in a species are called optimal codons, and those not utilized very often are classified as rare or low-usage codons. Codons can be substituted to reflect the preferred codon usage of the host, a process sometimes called “codon optimization” or “controlling for species codon bias.”

Optimized coding sequences containing codons preferred by a particular prokaryotic or eukaryotic host (see also, Murray et al. (1989) Nucl. Acids Res. 17:477-508) can be prepared, for example, to increase the rate of translation or to produce recombinant RNA transcripts having desirable properties, such as a longer half-life, as compared with transcripts produced from a non-optimized sequence. Translation stop codons can also be modified to reflect host preference. For example, typical stop codons for S. cerevisiae and mammals are UAA and UGA, respectively. The typical stop codon for monocotyledonous plants is UGA, whereas insects and E. coli commonly use UAA as the stop codon (Dalphin et al. (1996) Nucl. Acids Res. 24: 216-218). Methodology for optimizing a nucleotide sequence for expression in a plant is provided, for example, in U.S. Pat. No. 6,015,891, and the references cited therein.

A nucleic acid of the disclosure can be amplified using cDNA, mRNA or alternatively, genomic DNA, as a template and appropriate oligonucleotide primers according to standard PCR amplification techniques and those procedures described in the Examples section below. The nucleic acid so amplified can be cloned into an appropriate vector and characterized by DNA sequence analysis. Furthermore, oligonucleotides corresponding to nucleotide sequences can be prepared by standard synthetic techniques, e.g., using an automated DNA synthesizer.

It is also understood that an isolated nucleic acid molecule encoding a polypeptide homologous to the enzymes described herein can be created by introducing one or more nucleotide substitutions, additions or deletions into the nucleotide sequence encoding the particular polypeptide, such that one or more amino acid substitutions, additions or deletions are introduced into the encoded protein. Mutations can be introduced into the nucleic acid sequence by standard techniques, such as site-directed mutagenesis and PCR-mediated mutagenesis. In contrast to those positions where it may be desirable to make a non-conservative amino acid substitutions (see above), in some positions it is preferable to make conservative amino acid substitutions. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

In another embodiment a method for producing isopropanol is provided. The method includes culturing a recombinant microorganism as provided herein in the presence of a suitable substrate and under conditions suitable for the conversion of the substrate to isopropanol. The alcohol produced by a microorganism provided herein can be detected by any method known to the skilled artisan. Such methods include mass spectrometry. Culture conditions suitable for the growth and maintenance of a recombinant microorganism provided herein are described in the Examples below. The skilled artisan will recognize that such conditions can be modified to accommodate the requirements of each microorganism.

As previously discussed, general texts which describe molecular biological techniques useful herein, including the use of vectors, promoters and many other relevant topics, include Berger and Kimmel, Guide to Molecular Cloning Techniques, Methods in Enzymology Volume 152, (Academic Press, Inc., San Diego, Calif.) (“Berger”); Sambrook et al., Molecular Cloning—A Laboratory Manual, 2d ed., Vol. 1-3, Cold Spring Harbor Laboratory, Cold Spring Harbor, N.Y., 1989 (“Sambrook”) and Current Protocols in Molecular Biology, F. M. Ausubel et al., eds., Current Protocols, a joint venture between Greene Publishing Associates, Inc. and John Wiley & Sons, Inc., (supplemented through 1999) (“Ausubel”). Examples of protocols sufficient to direct persons of skill through in vitro amplification methods, including the polymerase chain reaction (PCR), the ligase chain reaction (LCR), Qβ-replicase amplification and other RNA polymerase mediated techniques (e.g., NASBA), e.g., for the production of the homologous nucleic acids of the disclosure are found in Berger, Sambrook, and Ausubel, as well as in Mullis et al. (1987) U.S. Pat. No. 4,683,202; Innis et al., eds. (1990) PCR Protocols: A Guide to Methods and Applications (Academic Press Inc. San Diego, Calif.) (“Innis”); Arnheim & Levinson (Oct. 1, 1990) C&EN 36-47; The Journal Of NIH Research (1991) 3: 81-94; Kwoh et al. (1989) Proc. Natl. Acad. Sci. USA 86: 1173; Guatelli et al. (1990) Proc. Nat'l. Acad. Sci. USA 87: 1874; Lomell et al. (1989) J. Clin. Chem. 35: 1826; Landegren et al. (1988) Science 241: 1077-1080; Van Brunt (1990) Biotechnology 8: 291-294; Wu and Wallace (1989) Gene 4:560; Barringer et al. (1990) Gene 89:117; and Sooknanan and Malek (1995) Biotechnology 13: 563-564. Improved methods for cloning in vitro amplified nucleic acids are described in Wallace et al., U.S. Pat. No. 5,426,039. Improved methods for amplifying large nucleic acids by PCR are summarized in Cheng et al. (1994) Nature 369: 684-685 and the references cited therein, in which PCR amplicons of up to 40 kb are generated. One of skill will appreciate that essentially any RNA can be converted into a double stranded DNA suitable for restriction digestion, PCR expansion and sequencing using reverse transcriptase and a polymerase. See, e.g., Ausubel, Sambrook and Berger, all supra.

Accordingly, the disclosure provides a recombinant microorganisms that produce isopropanol and include the expression or elevated expression of target enzymes such as a acetyl-coA acetyl transferase (e.g., atoB), an acetoacetyl-coA thiolase (e.g., thl), acetoacetyl-CoA transferase (e.g., atoAD), co-enzyme A transferase (e.g., ctfAB), acetoacetate decarboxylase (e.g., adc) and a secondary alcohol dehydrogenase (e.g., sadh), or any combination thereof, as compared to a parental microorganism. In addition, the microorganism may include a disruption, deletion or knockout of expression of an alcohol/acetoaldehyde dehydrogenase the preferentially uses acetyl-coA as a substrate (e.g. adhE gene), as compared to a parental microorganism. Other disruptions, deletions or knockouts can include one or more genes encoding a polypeptide or protein selected from the group consisting of: (i) an enzyme that catalyzes the NADH-dependent conversion of pyruvate to D-lactate; (ii) an enzyme that promotes catalysis of fumarate and succinate interconversion; (iii) an oxygen transcription regulator; (iv) an enzyme catalyzes the conversion of acetyl-coA to acetyl-phosphate; and (v) an enzyme that catalyzes the conversion of pyruvate to acetyl-coA and formate. In one aspect, the microorganism comprising a disruption, deletion or knockout of a combination of an alcohol/acetoaldehyde dehydrogenase and one or more of (i)-(iv) above, but not (v).

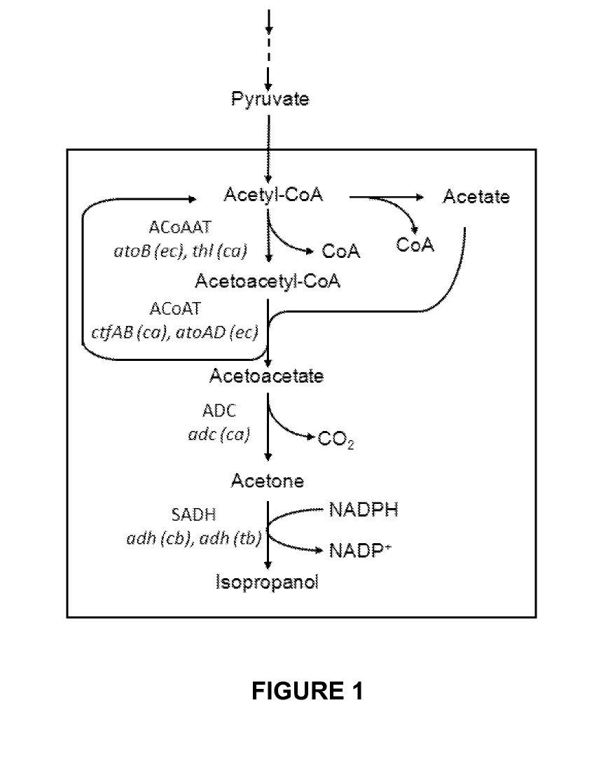

As depicted in FIG. 1, acetoacetyl-CoA can be produced by a recombinant microorganism metabolically engineered to express or over-express a thiolase or acetyl-CoA acetyltransferase.

Accordingly, a recombinant microorganism provided herein includes the elevated expression of at least one target enzyme, such as keto thiolase. In other aspects a recombinant microorganism can express a plurality of target enzymes involved in pathway to produce isopropanol from fermentation of a suitable carbon substrate. The plurality of enzymes can include a keto thiolase, a acetyl-CoA acetyltransferase, acetoacetyl-CoA transferase, a co-enzyme A transferase, acetoacetate decarboxylase and a secondary alcohol dehydrogenase, or any combination thereof.

As previously noted, the target enzymes described throughout this disclosure generally produce metabolites. For example, a keto thiolase produces acetoacetyl-CoA from a substrate that includes acetyl-CoA. In addition, the target enzymes described throughout this disclosure are encoded by polynucleotides. For example, a keto thiolase can be encoded by an atoB gene, polynucleotide or homolog thereof, or an fadA gene, polynucleotide or homolog thereof. The atoB gene or fadA gene can be derived from any biologic source that provides a suitable nucleic acid sequence encoding a suitable enzyme. For example, atoB gene or fadA gene can be derived from E. coli or C. acetobutylicum.

In another aspect, a recombinant microorganism provided herein includes elevated expression of an acetyl-CoA acetyltransferase as compared to a parental microorganism. The microorganism produces a metabolite that includes acetoacetyl-CoA from a substrate that includes acetyl-CoA. The acetyl-CoA acetyltransferase can be encoded by a thlA gene, polynucleotide or homolog thereof. The thlA gene or polynucleotide can be derived from the genus Clostridium.

In another aspect, a recombinant microorganism provided herein includes elevated expression of an acetoacetyl-CoA transferase as compared to a parental microorganism. The microorganism produces a metabolite that includes acetoacetate from a substrate that includes acetoacetyl-CoA. The acetoacetyl CoA transferase can be encoded by an atoAD gene, polynucleotide or homolog thereof. The atoAD can be derived from E. coli.

In another embodiment, a recombinant microorganism provided herein includes elevated expression of a co-enzyme A transferase compared to a parental microorganism. The microorganism produces a metabolite that includes acetoacetate from a substrate that includes acetoacetyl-CoA. The co-A transferase can be encoded by ctfAB gene, polynucleotide or homolog thereof. The ctfAB can be derived from Clostridium.

In another embodiment, a recombinant microorganism provided herein includes elevated expression of an acetoacetate decarboxylase compared to a parental microorganism. The microorganism produces a metabolite that includes acetone from a substrate that includes acetoacetate. The acetoacetate decarboxylase can be encoded by adc gene, polynucleotide or homolog thereof. The acetoacetate decarboxylase can be derived from Clostridium.

In another embodiment, a recombinant microorganism provided herein includes elevated expression of a secondary alcohol dehydrogenase compared to a parental microorganism. The microorganism produces a metabolite that includes isopropanol from a substrate that includes acetone. The secondary alcohol dehydrogenase can be encoded by a sadh gene, polynucleotide or homolog thereof. The secondary alcohol dehydrogenase can be derived from Clostridium or Thermoanaerobium.

The disclosure identifies specific genes useful in the methods, compositions and organisms of the disclosure; however it will be recognized that absolute identity to such genes is not necessary. For example, changes in a particular gene or polynucleotide comprising a sequence encoding a polypeptide or enzyme can be performed and screened for activity. Typically such changes comprise conservative mutation and silent mutations. Such modified or mutated polynucleotides and polypeptides can be screened for expression of a function enzyme activity using methods known in the art.

The following table and the disclosure provides non-limiting examples of genes and homologs for each gene having polynucleotide and polypeptide sequences available to the skilled person in the art.

| | Exemplary | | |
|---|---|---|---|
| Enzyme | Gene(s) | isopropanol | Exemplary Organism |
| acetyl-coA | atoB | + | E. Coli, |
| acetyltransferase | | | C. acetobutylicum |
| acetoacetyl-coA | thl, thlA, | + | E. coli, |
| thiolase | thlB | | C. acetobutylicum |
| Co-enzyme A | ctf, ctfA, | + | C. acetobutylicum |
| transferase | ctfB | | |
| Acetoacetyl-coA | ato, atoA, | + | E. coli |
| transferase | atoD | | |
| Acetoacetate | adc | + | C. acetobutylicum |
| decarboxylase | | | |
| Secondary alcohol | adh (sadh) | + | C. acetobutylicum, |
| dehydrogenase | | | T. brockii |

Exemplary yield data for E. coli comprising overexpression of the combination of C. acetobutylicum thl (acetyl-CoA acetyltransferase), E. coli atoAD (acetoacetyl-CoA transferase), C. acetobutylicum adc (acetoacetate decarboxylase) and C. beijerinckii adh (secondary alcohol dehydrogenase) produced 81.6 mM isopropanol in shake flasks with a yield of 43.5% (mol/mol) in the production phase.

The disclosure provides recombinant microorganism comprising a biosynthetic pathway that provides a yield of greater than wild-type microorganism. In one embodiment, the parental microorganism does not produced isopropanol. For example, wild-type E. coli do not produce traceable amounts of isopropanol. In yet another embodiment, the parental microorganism produced only trace amounts of isopropanol. In a specific embodiment the microorganism is an E. coli. In another aspect, a culture comprises a population microorganism that is substantially homogenous (e.g., from about 70-100% homogenous). In another aspect, a culture can comprises a combination of microorganism each having distinct biosynthetic pathways that produced metabolites that can be used by at least on other microorganism in culture in the production of isopropanol.

The disclosure provides accession numbers for various genes, homologs and variants useful in the generation of recombinant microorganism described herein. It is to be understood that homologs and variants described herein are exemplary and non-limiting. Additional homologs, variants and sequences are available to those of skill in the art using various databases including, for example, the National Center for Biotechnology Information (NCBI) access to which is available on the World-Wide-Web.

Ethanol Dehydrogenase (also referred to as Aldehyde-alcohol dehydrogenase) is encoded in E. coli by adhE. adhE comprises three activities: alcohol dehydrogenase (ADH); acetaldehyde/acetyl-CoA dehydrogenase (ACDH); pyruvate-formate-lyase deactivase (PFL deactivase); PFL deactivase activity catalyzes the quenching of the pyruvate-formate-lyase catalyst in an iron, NAD, and CoA dependent reaction. Homologs are known in the art (see, e.g., aldehyde-alcohol dehydrogenase (Polytomella sp. Pringsheim 198.80) gi|40644910|emb|CAD42653.2| (40644910); aldehyde-alcohol dehydrogenase (Clostridium botulinum A str. ATCC 3502) gi|148378348|ref|YP_{—}001252889.1| (148378348); aldehyde-alcohol dehydrogenase (Yersinia pestis C092) gi|16122410|ref|NP_{—}405723.1| (16122410); aldehyde-alcohol dehydrogenase (Yersinia pseudotuberculosis IP 32953) gi|51596429|ref|YP_{—}070620.1| (51596429); aldehyde-alcohol dehydrogenase (Yersinia pestis C092) gi|115347889|emb|CAL20810.1| (115347889); aldehyde-alcohol dehydrogenase (Yersinia pseudotuberculosis IP 32953) gi|51589711|emb|CAH21341.1| (51589711); Aldehyde-alcohol dehydrogenase (Escherichia coli CFT073) gi|26107972|gb|AAN80172.1|AE016760_{—}31(26107972); aldehyde-alcohol dehydrogenase (Yersinia pestis biovar Microtus str. 91001) gi|45441777|ref|NP_{—}993316.1| (45441777); aldehyde-alcohol dehydrogenase (Yersinia pestis biovar Microtus str. 91001) gi|45436639|gb|AAS62193.1| (45436639); aldehyde-alcohol dehydrogenase (Clostridium perfringens ATCC 13124) gi|110798574|ref|YP_{—}697219.1| (110798574); aldehyde-alcohol dehydrogenase (Shewanella oneidensis MR-1)gi|24373696|ref|NP_{—}717739.1| (24373696); aldehyde-alcohol dehydrogenase (Clostridium botulinum A str. ATCC 19397) gi|153932445|ref|YP_{—}001382747.1| (153932445); aldehyde-alcohol dehydrogenase (Yersinia pestis biovar Antigua str. E1979001) gi|165991833|gb|EDR44134.1| (165991833); aldehyde-alcohol dehydrogenase (Clostridium botulinum A str. Hall) gi|153937530|ref|YP_{—}001386298.1| (153937530); aldehyde-alcohol dehydrogenase (Clostridium perfringens ATCC 13124) gi|110673221|gb|ABG82208.1| (110673221); aldehyde-alcohol dehydrogenase (Clostridium botulinum A str. Hall) gi|152933444|gb|ABS38943.1| (152933444); aldehyde-alcohol dehydrogenase (Yersinia pestis biovar Orientalis str. F1991016) gi|165920640|gb|EDR37888.1| (165920640); aldehyde-alcohol dehydrogenase (Yersinia pestis biovar Orientalis str. IP275)gi|165913933|gb|EDR32551.1| (165913933); aldehyde-alcohol dehydrogenase (Yersinia pestis Angola) gi|162419116|ref|YP_{—}001606617.1| (162419116); aldehyde-alcohol dehydrogenase (Clostridium botulinum F str. Langeland) gi|153940830|ref|YP_{—}001389712.1| (153940830); aldehyde-alcohol dehydrogenase (Escherichia coli HS) gi|157160746|ref|YP_{—}001458064.1 | (157160746); aldehyde-alcohol dehydrogenase (Escherichia coli E24377A) gi|157155679|ref|YP_{—}001462491.1| (157155679); aldehyde-alcohol dehydrogenase (Yersinia enterocolitica subsp. enterocolitica 8081) gi|123442494|ref|YP_{—}001006472.1| (123442494); aldehyde-alcohol dehydrogenase (Synechococcus sp. JA-3-3Ab) gi|86605191|ref|YP_{—}473954.1| (86605191); aldehyde-alcohol dehydrogenase (Listeria monocytogenes str. 4b F2365) gi|46907864|ref|YP_{—}014253.1| (46907864); aldehyde-alcohol dehydrogenase (Enterococcus faecalis V583) gi|29375484|ref|NP_{—}814638.1| (29375484); aldehyde-alcohol dehydrogenase (Streptococcus agalactiae 2603V/R) gi|22536238|ref|NP_{—}687089.1| (22536238); aldehyde-alcohol dehydrogenase (Clostridium botulinum A str. ATCC 19397) gi|152928489|gb|ABS33989.1| (152928489); aldehyde-alcohol dehydrogenase (Escherichia coli E24377A) gi|157077709|gb|ABV17417.1| (157077709); aldehyde-alcohol dehydrogenase (Escherichia coli HS) gi|157066426|gb|ABV05681.1| (157066426); aldehyde-alcohol dehydrogenase (Clostridium botulinum F str. Langeland) gi|152936726|gb|ABS42224.1| (152936726); aldehyde-alcohol dehydrogenase (Yersinia pestis CA88-4125) gi|149292312|gb|EDM42386.1| (149292312); aldehyde-alcohol dehydrogenase (Yersinia enterocolitica subsp. enterocolitica 8081) gi|122089455|emb|CAL12303.1| (122089455); aldehyde-alcohol dehydrogenase (Chlamydomonas reinhardtii) gi|92084840|emb|CAF04128.1| (92084840); aldehyde-alcohol dehydrogenase (Synechococcus sp. JA-3-3Ab) gi|86553733|gb|ABC98691.1| (86553733); aldehyde-alcohol dehydrogenase (Shewanella oneidensis MR-1) gi|24348056|gb|AAN55183.1|AE015655_{—}9(24348056); aldehyde-alcohol dehydrogenase (Enterococcus faecalis V583) gi|29342944|gb|AA080708.1| (29342944); aldehyde-alcohol dehydrogenase (Listeria monocytogenes str. 4b F2365) gi|46881133|gb|AAT04430.1| (46881133); aldehyde-alcohol dehydrogenase (Listeria monocytogenes str. 1/2a F6854) gi|47097587|ref|ZP_{—}00235115.1| (47097587); aldehyde-alcohol dehydrogenase (Listeria monocytogenes str. 4b H7858) gi|47094265|ref|ZP_{—}00231973.1| (47094265); aldehyde-alcohol dehydrogenase (Listeria monocytogenes str. 4b H7858) gi|47017355|gb|EAL08180.1| (47017355); aldehyde-alcohol dehydrogenase (Listeria monocytogenes str. 1/2a F6854) gi|47014034|gb|EAL05039.1| (47014034); aldehyde-alcohol dehydrogenase (Streptococcus agalactiae 2603V/R) gi|22533058|gb|AAM98961.1|AE014194_{—}6(22533058)p; aldehyde-alcohol dehydrogenase (Yersinia pestis biovar Antigua str. E1979001) gi|166009278|ref|ZP_{—}02230176.11(166009278); aldehyde-alcohol dehydrogenase (Yersinia pestis biovar Orientalis str. IP275) gi|165938272|ref|ZP_{—}02226831.1| (165938272); aldehyde-alcohol dehydrogenase (Yersinia pestis biovar Orientalis str. F1991016) gi|165927374|ref|ZP_{—}02223206.1| (165927374); aldehyde-alcohol dehydrogenase (Yersinia pestis Angola) gi|162351931|gb|ABX85879.1| (162351931); aldehyde-alcohol dehydrogenase (Yersinia pseudotuberculosis IP 31758) gi|153949366|ref|YP_{—}001400938.1| (153949366); aldehyde-alcohol dehydrogenase (Yersinia pseudotuberculosis IP 31758) gi|152960861|gb|ABS48322.1| (152960861); aldehyde-alcohol dehydrogenase (Yersinia pestis CA88-4125) gi|149365899|ref|ZP_{—}01887934.1| (149365899); Acetaldehyde dehydrogenase (acetylating) (Escherichia coli CFT073) gi|26247570|ref|NP_{—}753610.1| (26247570); aldehyde-alcohol dehydrogenase (includes: alcohol dehydrogenase; acetaldehyde dehydrogenase (acetylating) (EC 1.2.1.10) (acdh); pyruvate-formate-lyase deactivase (pfl deactivase)) (Clostridium botulinum A str. ATCC 3502) gi|148287832|emb|CAL81898.1| (148287832); aldehyde-alcohol dehydrogenase (Includes: Alcohol dehydrogenase (ADH); Acetaldehyde dehydrogenase (acetylating) (ACDH); Pyruvate-formate-lyase deactivase (PFL deactivase)) gi|71152980|sp|P0A9Q7.2|ADHE_ECOLI(71152980); aldehyde-alcohol dehydrogenase (includes: alcohol dehydrogenase and acetaldehyde dehydrogenase, and pyruvate-formate-lyase deactivase (Erwinia carotovora subsp. atroseptica SCRI1043) gi|50121254|ref|YP_{—}050421.1| (50121254); aldehyde-alcohol dehydrogenase (includes: alcohol dehydrogenase and acetaldehyde dehydrogenase, and pyruvate-formate-lyase deactivase (Erwinia carotovora subsp. atroseptica SCRI1043) gi|49611780|emb|CAG75229.1| (49611780); Aldehyde-alcohol dehydrogenase (Includes: Alcohol dehydrogenase (ADH); Acetaldehyde dehydrogenase (acetylating) (ACDH)) gi|19858620|sp|P33744.3|ADHE_CLOAB(19858620); Aldehyde-alcohol dehydrogenase (Includes: Alcohol dehydrogenase (ADH); Acetaldehyde dehydrogenase (acetylating) (ACDH); Pyruvate-formate-lyase deactivase (PFL deactivase)) gi|71152683|sp|P0A9Q8.2|ADHE_ECO57(71152683); aldehyde-alcohol dehydrogenase (includes: alcohol dehydrogenase; acetaldehyde dehydrogenase (acetylating); pyruvate-formate-lyase deactivase (Clostridium difficile 630) gi|126697906|ref|YP_{—}001086803.1| (126697906); aldehyde-alcohol dehydrogenase (includes: alcohol dehydrogenase; acetaldehyde dehydrogenase (acetylating); pyruvate-formate-lyase deactivase (Clostridium difficile 630) gi|115249343|emb|CAJ67156.1| (115249343); Aldehyde-alcohol dehydrogenase (includes: alcohol dehydrogenase (ADH) and acetaldehyde dehydrogenase (acetylating) (ACDH); pyruvate-formate-lyase deactivase (PFL deactivase)) (Photorhabdus luminescens subsp. laumondii TT01) gi|37526388|ref|NP_{—}929732.1| (37526388); aldehyde-alcohol dehydrogenase 2 (includes: alcohol dehydrogenase; acetaldehyde dehydrogenase) (Streptococcus pyogenes str. Manfredo) gi|134271169|emb|CAM29381.1| (134271169); Aldehyde-alcohol dehydrogenase (includes: alcohol dehydrogenase (ADH) and acetaldehyde dehydrogenase (acetylating) (ACDH); pyruvate-formate-lyase deactivase (PFL deactivase)) (Photorhabdus luminescens subsp. laumondii TTO1) gi|36785819|emb|CAE14870.1| (36785819); aldehyde-alcohol dehydrogenase (includes: alcohol dehydrogenase and pyruvate-formate-lyase deactivase (Clostridium difficile 630) gi|126700586|ref|YP_{—}001089483.1| (126700586); aldehyde-alcohol dehydrogenase (includes: alcohol dehydrogenase and pyruvate-formate-lyase deactivase (Clostridium difficile 630) gi|115252023|emb|CAJ69859.1| (115252023); aldehyde-alcohol dehydrogenase 2 (Streptococcus pyogenes str. Manfredo) gi|139472923|ref|YP_{—}001127638.1| (139472923); aldehyde-alcohol dehydrogenase E (Clostridium perfringens str. 13) gi|18311513|ref|NP_{—}563447.1| (18311513); aldehyde-alcohol dehydrogenase E (Clostridium perfringens str. 13) gi|18146197|dbj|BAB82237.1| (18146197); Aldehyde-alcohol dehydrogenase, ADHE1 (Clostridium acetobutylicum ATCC 824) gi|15004739|ref|NP_{—}149199.1| (15004739); Aldehyde-alcohol dehydrogenase, ADHE1 (Clostridium acetobutylicum ATCC 824) gi|14994351|gb|AAK76781.1|AE001438_{—}34(14994351); Aldehyde-alcohol dehydrogenase 2 (Includes: Alcohol dehydrogenase (ADH); acetaldehyde/acetyl-CoA dehydrogenase (ACDH)) gi|2492737|sp|Q24803.1|ADH2_ENTHI(2492737); alcohol dehydrogenase (Salmonella enterica subsp. enterica serovar Typhi str. CT18) gi|16760134|ref|NP_{—}455751.1| (16760134); and alcohol dehydrogenase (Salmonella enterica subsp. enterica serovar Typhi) gi|16502428|emb|CAD08384.1| (16502428)), each sequence associated with the accession number is incorporated herein by reference in its entirety.

An acetoacetyl-coA thiolase (also sometimes referred to as an acetyl-coA acetyltransferase) catalyzes the production of acetoacetyl-coA from two molecules of acetyl-coA. Depending upon the organism used a heterologous acetoacetyl-coA thiolase (acetyl-coA acetyltransferase) can be engineered for expression in the organism. Alternatlively a native acetoacetyl-coA thiolase (acetyl-coA acetyltransferase) can be overexpressed. Acetoacetyl-coA thiolase is encoded in E. coli by thl. Acetyl-coA acetyltransferase is encoded in C. acetobutylicum by atoB. THL and AtoB homologs and variants are known. For examples, such homologs and variants include, for example, acetyl-coa acetyltransferase (thiolase) (Streptomyces coelicolor A3(2)) gi|21224359|ref|NP_{—}630138.1| (21224359); acetyl-coa acetyltransferase (thiolase) (Streptomyces coelicolor A3(2)) gi|3169041|emb|CAA19239.1| (3169041); Acetyl CoA acetyltransferase (thiolase) (Alcanivorax borkumensis SK2) gi|110834428|ref|YP_{—}693287.1| (110834428); Acetyl CoA acetyltransferase (thiolase) (Alcanivorax borkumensis SK2) gi|110647539|emb|CAL17015.1| (110647539); acetyl CoA acetyltransferase (thiolase) (Saccharopolyspora erythraea NRRL 2338) gi|133915420|emb|CAM05533.11(133915420); acetyl-coa acetyltransferase (thiolase) (Saccharopolyspora erythraea NRRL 2338) gi|134098403|ref|YP_{—}001104064.1| (134098403); acetyl-coa acetyltransferase (thiolase) (Saccharopolyspora erythraea NRRL 2338) gi|133911026|emb|CAM01139.1| (133911026); acetyl-CoA acetyltransferase (thiolase) (Clostridium botulinum A str. ATCC 3502) gi|1482906321emb|CAL84761.1| (148290632); acetyl-CoA acetyltransferase (thiolase) (Pseudomonas aeruginosa UCBPP-PA14) gi|115586808|gb|ABJ12823.1| (115586808); acetyl-CoA acetyltransferase (thiolase) (Ralstonia metallidurans CH34) gi|93358270|gb|ABF12358.1| (93358270); acetyl-CoA acetyltransferase (thiolase) (Ralstonia metallidurans CH34) gi|93357190|gb|ABF11278.1| (93357190); acetyl-CoA acetyltransferase (thiolase) (Ralstonia metallidurans CH34) gi|93356587|gb|ABF10675.1| (93356587); acetyl-CoA acetyltransferase (thiolase) (Ralstonia eutropha JMP134) gi|72121949|gb|AAZ64135.1| (72121949); acetyl-CoA acetyltransferase (thiolase) (Ralstonia eutropha JMP134)gi|72121729|gb|AAZ63915.1| (72121729); acetyl-CoA acetyltransferase (thiolase) (Ralstonia eutropha JMP134) gi|72121320|gb|AAZ63506.1| (72121320); acetyl-CoA acetyltransferase (thiolase) (Ralstonia eutropha JMP134) gi|7212100|gb|AAZ63187.1| (72121001); acetyl-CoA acetyltransferase (thiolase) (Escherichia coli) gi|2764832|emb|CAA66099.1| (2764832), each sequence associated with the accession number is incorporated herein by reference in its entirety.

A co-enzyme A transferase catalyzes the production of acetoacetate from acetoacetyl-coA. Depending upon the organism used a heterologous co-enzyme A transferase can be engineered for expression in the organism. Alternatively a native co-enzyme A transferase can be overexpressed. Co-enzyme A transferase is encoded in C. acetobutylicum by ctfAB and ctfAB homologs and variants are known. For examples, such homologs and variants include, for example, Co-enzyme A transferase (C. acetobutylicum adhE, ctfA and ctfB genes) gi|2980801emb|X72831.1| (298080); Clostridium acetobutylicum ATCC 824 megaplasmid pSOL1 gi|14994317|gb|AE001438.3| (14994317), each sequence associated with the accession number is incorporated herein by reference in its entirety.

An acetoacetyl coA transferase catalyzes the production of acetoacetate from acetoacetyl-coA. Depending upon the organism used a heterologous acetoacetyl coA transferase can be engineered for expression in the organism. Alternatively a native acetoacetyl coA transferase can be overexpressed. Acetoacetyl coA transferase is encoded in E. coli by atoAB. AtoAB homologs and variants are known. For examples, such homologs and variants include (or can be found in the genome sequence of), for example, NC_{—}010468 Escherichia coli ATCC 8739, gi|170018061|ref|NC_{—}010468.1| (170018061); NC 009654 Marinomonas sp. MWYL1 gi|152994043|ref|NC_{—}009654.1| (152994043); NC_{—}009454; Pelotomaculum thermopropionicum SI, complete genome gi|147676335|ref|NC_{—}009454.1| (147676335); Escherichia coli APEC O1, complete genome gi|117622295|ref|NC_{—}008563.1|(117622295); Escherichia coli 536, complete genome gi|110640213|ref|NC_{—}008253.1| (110640213); Escherichia coli UTI89, complete genome gi|91209055|ref|NC_{—}007946.1| (91209055); Escherichia coli HS, complete genome gi|157159467|ref|NC_{—}009800.1| (157159467); Clostridium beijerinckii NCIMB 8052, complete genome gi|150014892|ref|NC_{—}009617.1| (150014892); Shigella sonnei Ss046, complete genome gi|74310614|ref|NC_{—}007384.1| (74310614) Citrobacter koseri ATCC BAA-895, complete genome gi|157144296|ref|NC_{—}009792.1| (157144296); Rhodospirillum rubrum ATCC 11170, complete genome gi|83591340|ref|NC_{—}007643.1| (83591340); Escherichia coli CFT073, complete genome gi|26245917|ref|NC_{—}004431.1| (26245917); Salmonella typhimurium LT2, complete genome gi|16763390|ref|NC_{—}003197.1| (16763390); Escherichia coli ATCC 8739, complete genome gi|169752989|gb|CP000946.1| (169752989); Escherichia coli str. K-12 substr. MG1655, complete genome gi|49175990|ref|NC_{—}000913.2| (49175990); Escherichia coli str. K-12 substr. MG1655, complete genome gi|48994873|gb|U00096.2| (48994873); Marinomonas sp. MWYL1, complete genome gi|150834967|gb|CP000749.1| (150834967); Clostridium beijerinckii NCIMB 8052, complete genome gi|149901357|gb|CP000721.1| (149901357); each sequence associated with the accession number is incorporated herein by reference in its entirety.

An acetoacetate decarboxylase catalyzes the decarboxylation of acetoacetate forming acetone and carbon dioxide. Depending upon the organism used a heterologous acetoacetate decarboxylase can be engineered for expression in the organism. Alternatively a native acetoacetate decarboxylase can be overexpressed. Acetoacetate decarboxylase is encoded in Clostridium acetobutylicum by acd. ACD homologs and variants are known. A sequence for acd is set forth in gi|15004705|ref|NC_{—}001988.2| (15004705) Clostridium acetobutylicum ATCC 824 plasmid pSOL1; each sequence associated with the accession number is incorporated herein by reference in its entirety.

A secondary alcohol dehydrogenase catalyzes the reduction of methyl ketones to their corresponding 2-alcohols in the presence of NADH. Depending upon the organism used a heterologous secondary alcohol dehydrogenase can be engineered for expression in the organism. Alternatively a native secondary alcohol dehydrogenase can be overexpressed. A secondary alcohol dehydrogenase useful in the methods and compositions of the disclosurue is encoded in Clostridium acetobutylicum or E. coli by adh (sadh). SADH homologs and variants are known. For example, Rhodococcus ruber, sadh gene and sudh gene are set forth in accession number gi|21615551|emb|AJ491307.1|(21615551); Candida metapsilosis partial sadh gene for secondary alcohol dehydrogenase, type strain 96144 gi|47826366|emb|AJ698115.1| (47826366); and Candida parapsilosis gene for SADH, complete cds gi|2815408|dbj|AB010636.11(2815408); each sequence associated with the accession number is incorporated herein by reference in its entirety.

Culture conditions suitable for the growth and maintenance of a recombinant microorganism provided herein are described in the Examples below. The skilled artisan will recognize that such conditions can be modified to accommodate the requirements of each microorganism. Appropriate culture conditions useful in producing a isopropanol product comprise conditions of culture medium pH, ionic strength, nutritive content, etc.; temperature; oxygen/CO₂/nitrogen content; humidity; and other culture conditions that permit production of the compound by the host microorganism, i.e., by the metabolic action of the microorganism. Appropriate culture conditions are well known for microorganisms that can serve as host cells.

In one embodiment a microorganism of the disclosure can be characterized as an E. coli comprising rrnBT14DlacZWJ16 hsdR514 DaraBADAH33 DrhaBADLD78 (with F′ transduced from XL-1 blue to supply lacIq), (atoB-ctfAB-adc), pTA30 (atoB-atoAD-adc), pTA39 (thl-atoAD-adc), and pTA41 (thl-ctfAB-adc).

### EXAMPLES

Several species of Clostridium have been evaluated for isopropanol production, including 52 strains of C. beijerinckii. The maximum production of isopropanol from these strains was 30 mM. Limited knowledge about metabolic regulation of these strains and the difficulty of gene manipulation have hindered further improvements in isopropanol production. On the other hand, E. coli is one of most studied and easily manipulated organisms for metabolic engineering. This bacterium has already been shown to produce high titers of ethanol and many other biochemicals.

Bermejo et al. (Appl. Environ. Microbiol. 64:1079-1085, 1998) have produced acetone in E. coli by introducing four genes from C. acetobutylicum ATCC824 (thl, ctfAB, and adc coding acetyl-CoA acetyltransferase, acetoacetyl-CoA-transferase, and acetoacetate decarboxylase, respectively) under the control of the thl promoter from C. acetobutylicum. This engineered E. coli strain produced almost the same level of acetone as C. acetobutylicum ATCC 824. However, isopropanol production in E. coli has not been reported. The disclosure provides an engineered a synthetic pathway for the production of isopropanol in microorganisms including E. coli.

The strategy for the biosynthesis of isopropanol in utilizes the pathway modeled after C. beijerinckii, which produces isopropanol from acetyl-coenzyme A (acetyl-CoA) via acetone (FIG. 1). First, an acetyl-CoA acetyltransferase condenses two molecules of acetyl-CoA to one molecule of acetoacetyl-CoA. Next, an acetoacetyl-CoA transferase transfers CoA from acetoacetyl-CoA to acetate or to butyrate, forming acetoacetate which is then converted to acetone and CO₂ by an acetoacetate decarboxylase. A primary-secondary alcohol dehydrogenase (hereafter referred to as the secondary alcohol dehydrogenase, SADH) converts acetone to isopropanol in an NADPH-dependent reaction. To optimize the pathway, native E. coli acetyl-CoA acetyltransferase (encoded by atoB) and acetoacetyl-CoA transferase (encoded by atoAD) were examined, in performing the initial steps of acetone production. Furthermore, the activity of the SADH (encoded by adh) from C. beijerinckii NRRL B593 and T. brockii HTD4 were compared in performing the final step of isopropanol production. These two SADH's have previously been expressed and characterized in E. coli.

Table 1 shows the strains and plasmids used in this study. E. coli B strain (ATCC 11303) with lacI^{g} introduced from E. coli DH5αZ1 by P1 transduction was used as the host strain and designated as TA11. Multiple combinations of the acetone pathway genes thl, ctfAB, atoB, atoAD, and adc were cloned into a ColE1-deliverd plasmid (pSA40) under the control of P_{L}lacO₁ promoter. The resulting plasmids were named pTA29 (atoB-ctfAB-adc), pTA30 (atoB-atoAD-adc), pTA39 (thl-atoAD-adc), and pTA41 (thl-ctfAB-adc) (see Table 1 for details). The adh gene from C. beijerinckii or T. brockii was cloned into a p15A-delivered vector (pZA31-luc) under the control of P_{L}lacO₁ to generate plasmids pTA36 and pTA18, respectively. Each combination of the acetone pathway genes and the isopropanol-producing genes was introduced into TA11 for evaluation.

The same ribosome binding site (RBS) (GAAGGAGATATACAT (SEQ ID NO:11) was used for all genes of isopropanol production pathway except for the atoB and thl genes which were cloned downstream of the P_{L}lacO₁ promoter using the expression construct's RBS. The source of used RBS is pET-31b(+) plasmid (Invitrogen, Carlsbad, Calif.). All genes except the two adh genes were PCR-amplified from chromosomal DNA and sequence verified. The two adh genes were synthesized by Epoch Biolabs (Houston, Tex.) after codon optimization for E. coli. The following conditions were used for optimization. 15% cut off was used for codon efficiency: any codon below 15% was removed except for positions with strong secondary structures (in this case codons of lower frequency were used to alleviate the problem). Secondary structure was checked using a build-in M-fold module. Stem loop and pseudo Shine-Dalgarno sequence were avoided. The number changed base pairs by this optimization is about 290 for C. beijerinckii and 240 for T. brockii. Restriction enzymes, phosphatase (New England Biolabs, Ipswich, Mass.), ligase (rapid DNA ligation kit; Roche, Manheim, Germany), And DNA polymerase (KOD DNA polymerase, EMD Chemicals, San Diego, Calif.) were used for cloning. Oligonucleotides were synthesized by Invitrogen (Carlsbad, Calif.).

pTA29 (P_{L}lacO₁:: atoB-ctfAB-adc)- To replace P_{L}tetO₁ of pZE21-MCS1 with P_{L}lacO₁, pZE12-luc was digested with AatII and Acc65I. The shorter fragment was purified and cloned into plasmid pZE21-MCS1 cut with the same enzymes, creating pSA40. To clone atoB, ctfAB and adc, Acc65I-SalI (atoB), SalI-XmaI (ctfAB) and XmaI-BamHI (adc) recognition sites in pSA40 were used. AatII and AvrII recognition sites were used to replace the kanamycin resistance gene with the ampicillin resistance gene. To clone atoB, genomic DNA of E. coli K-12 MG1655 was used as template with a pair of primers TA15F (5′ CGCGGTACCATGAAAAATTGTGTCATCGTCAGTG 3′ (SEQ ID NO:12)) and TA16R (5′ CCGCGTCGACTTAATTCAACCGTTCAATCACCATC 3′ (SEQ ID NO:13)) and PCR products were digested with Acc65I and SalI. To clone ctfAB, genomic DNA of C. acetobutylicum ATCC824 (ATCC) was used as template with a pair of primers TA19F (5′ CCGCGTCGACGAAGGAGATATACATATGAACTCTAAAATAATTAGATTTG 3′ (SEQ ID NO:14)) and TA4R (5′ CGCCCCGGGCTAAACAGCCATGGGTCTAAGTTCA 3′ (SEQ ID NO:15)) and PCR products were digested with SalI and XmaI. To clone adc, genomic DNA of C. acetobutylicum ATCC824 was used as template with a pair of primers TA20F (5′ CGCCCCGGGGAAGGAGATATACATATGTTAAAGGATGAAGTAATTAAAC 3′ (SEQ ID NO:16)) and TA6R (5′ CGCGGATCCTTACTTAAGATAATCATATATAACT 3′ (SEQ ID NO:17)) and PCR products were digested with XmaI and BamHI.

pTA30 (P_{L}lacO₁:: atoB-atoAD-adc)- To clone atoAD, genomic DNA of E. coli K-12 MG1655 was used as template with a pair of primers TA21F (5′ CCGCGTCGACGAAGGAGATATACATATGAAAACAAA ATTGATGACATTAC 3′ (SEQ ID NO:18)) and TA18R (5′ CGCCCCGGGTC ATAAATCACCCCGTTGCGTATTC 3′ (SEQ ID NO:19)). PCR products were digested with SalI and XmaI and cloned into plasmid pTA29 cut with the same enzymes.

pTA39 (P_{L}lacO₁:: thl-atoAD-adc)- To clone thl, genomic DNA of C. acetobutylicum ATCC824 was used as template with a pair of primers TA13F (5′ CGCGGTACCATGAAAGAAGTTGTA ATAGCTAGTG 3′ (SEQ ID NO:20)) and TA14R (5′ CCGCGTCGACCTAG CACTTTTCTAGCAATATTGC 3′ (SEQ ID NO:21)). PCR products were digested with Acc65I and SalI and cloned into plasmid pTA30 cut with the same enzymes.

pTA41 (P_{L}lacO₁:: thl-ctfAB-adc)- To clone thl, genomic DNA of C. acetobutylicum ATCC824 was used as template with a pair of primers TA13F and TA14R. PCR products were digested with Acc65I and SalI and cloned into plasmid pTA29 cut with the same enzymes.

pTA18 (P_{L}lacO₁:: adh (T. brockii))- To clone adh (T. brockii), synthesized DNA of T. brockii HTD4 (Epoch Biolabs) was used as a template with a pair of primers TA7F (5′ CGCGGTACCATGAAAGGTTTTGCAATGCTGTCCA 3′ (SEQ ID NO:22)) and TA8R (5′ CGCTCTAGACTATGCTAAAATCACCACTGGTTTAATT 3′ (SEQ ID NO:23)). PCR products were digested with Acc65I and XbaI and cloned into plasmid pZE12-luc cut with the same enzymes, creating pTA8. To replace replication origin and the ampicillin resistance gene with p15A and the kanamycin resistance gene, pZA31-luc was digested with AatII and AvrII. The shorter fragment was purified and cloned into pTA8 cut with the same enzymes to create pTA18. The codon usage of the synthesized DNA was optimized for expression in E. coli and stem loop structures were avoided by checking with a secondary structure prediction program. This sequence is shown in supplemental material.

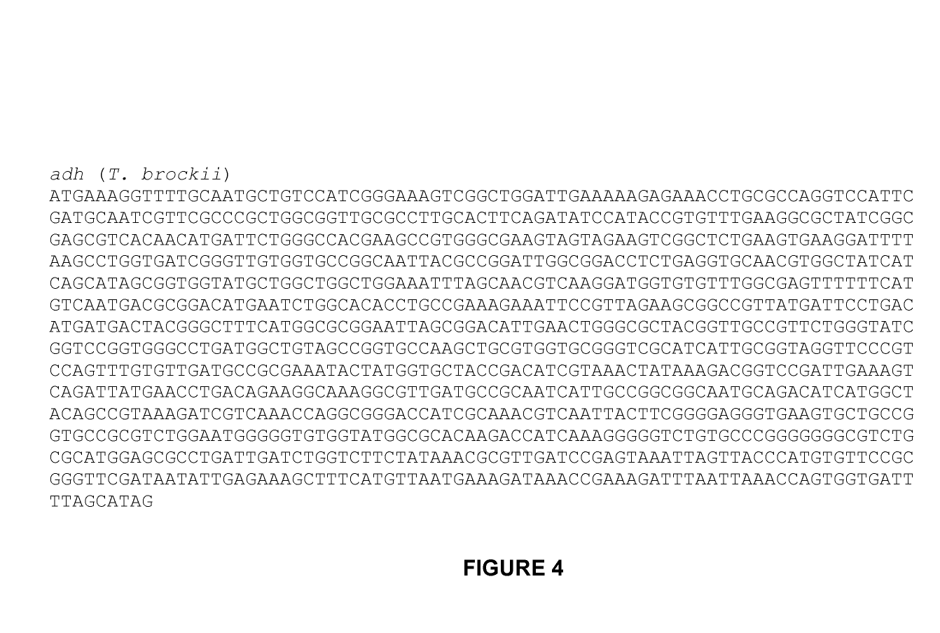

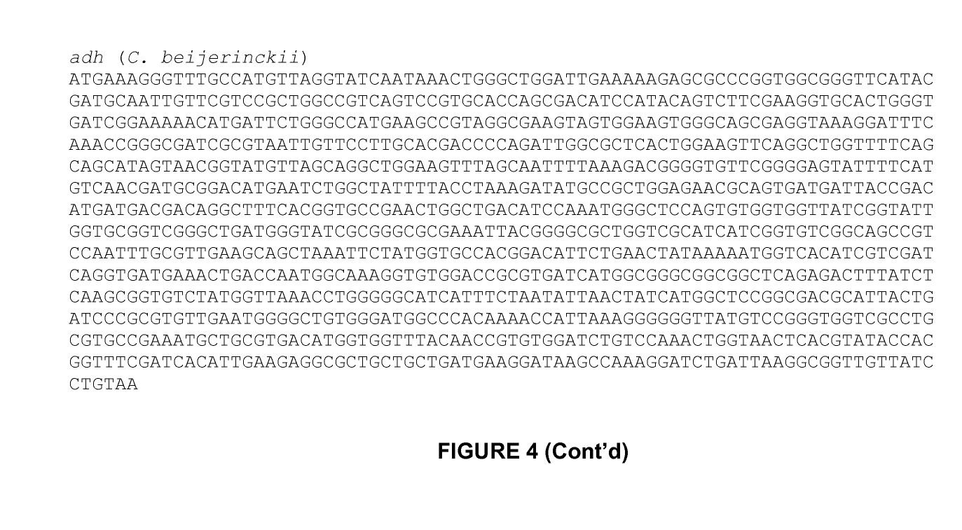

pTA36 (P_{L}lacO₄:: adh (C. beijerinckii))- To clone adh (C. beijerinckii), the plasmid with synthesized DNA of C. beijerinckii NRRL B593 (Epoch Biolabs) was digested with Acc65I and SalI and cloned into plasmid pZE12-luc cut with the same enzymes, creating pTA34. To replace replication origin and the ampicillin resistance gene with p15A and the kanamycin resistance gene, pZA31-luc was digested with AatII and AvrII. The shorter fragment was purified and cloned into plasmid the plasmid cut with the same enzymes to create pTA36. The codon usage of the synthesized DNA was optimized in the same way as the adh gene from T. brockii (see, e.g., FIG. 4).

As the preculture medium, SD-7 containing 2% glucose was prepared (NH₄Cl, 7.0 g/l; KH₂PO₄, 1.5 g/l; Na₂HPO₄, 1.5 g/l; K₂SO₄, 0.35 g/l; MgSO₄O.7H₂O, 0.17 g/l, Trace elements, 0.8 ml/l; Yeast extract; 5 g/l) as described (13). SD-8 (NH₄Cl, 7.0 g/l; KH₂PO₄, 7.5 g/l; Na₂HPO₄, 7.5 g/l; K₂SO₄, 0.85, MgSO₄O.7H₂O, 0.17 g/l, Trace elements, 0.8 ml/l; Yeast extract; 10 g/l) (13) medium contain 2% glucose was used for fermentations. Trace element solution contained the following (in grams per liter of 5 M HCl): FeSO₄O.7H₂O, 40.0; MnSO₄.H₂O, 10.0; Al₂(SO₄)₃, 28.3; CoCl₂.6H₂O, 4.0; ZnSO₄O.7H₂O, 2.0; Na₂MoO₄.2H₂O; CuCl₂.2H₂O, 1.0; and H₃BO₄, 0.5. Antibiotics were added as appropriate; ampicillin 100 μg/ml and chloramphenicol 40 μg/ml.

Preculture containing 5 ml of SD-7 medium in test tube was performed at 37° C. overnight (17 h) on rotary shaker (250 rpm). 250 μl of overnight culture was inoculated into 25 ml of SD-8 medium in 250 ml flask with baffles. When OD₆₀₀ was ˜1.0 (3 h), 0.1 mM IPTG was added for induction. Cells were grown at 37° C. for 30.5 h on a rotary shaker (250 rpm) and harvested at 0, 3, 6.5, 9.5, 12, 24, 28.5 and 30.5 h.

Glucose was measured using a glucose analysis reagent (Sigma Aldrich). Various alcohols were quantified by gas chromatography with a flame ionization detector. The system consisted of model 5890A gas chromatograph (Hewlett Packard, Avondale, Pa.) and a model 7673A automatic injector, sampler and controller (Hewlett Packard). The separation of alcohol compounds was carried out by A DB-WAX capillary column (30 m, 0.32 mm-i.d., 0.50 μm-film thickness; Agilent Technologies). GC oven temperature was initially held at 40° C. for 5 min and raised with a gradient of 15° C./min until 120° C. followed by a gradient 50° C./min until 230° C. and held for 4 min. Helium was used as the carrier gas with 9.3 psi inlet pressure. The injector and detector were maintained at 225° C. 0.5 μl supernatant of culture broth was injected in split injection:mode (1:15 split ratio). 1-propanol was used as the internal standard.

For other secreted metabolites, filtered supernatant was applied (20 μl) to an Agilent 1100 HPLC equipped with an auto-sampler (Agilent Technologies) and a BioRad (Biorad Laboratories, Hercules, Calif.) Aminex HPX87 column (5 mM H₂SO₄, 0.6 ml/min, column temperature at 65° C.). Organic acids (fumarate, lactate, citrate, pyruvate, formate, malate, acetate, and succinate) were detected using a photodiode array detector at 210 nm.

Cells harvested at 4 h after induction were used for enzyme assays after centrifugation. Crude extract in 50 mM Tris chloride (pH8) was prepared under anaerobic condition with 0.1 mm glass beads and Mini Bead Beater 8 (Biospec Product, OK). SADH activities were measured by following the reduction of acetone (6.7 mM) with NADPH at 25° C. under Ar. The assay mixture contained 50 mM Tris-Cl buffer (pH 7.5), 1 mM dithiothreitol, and 0.2 mM NADPH. One unit of activity is the oxidation of 1 μmol of NADPH per min.

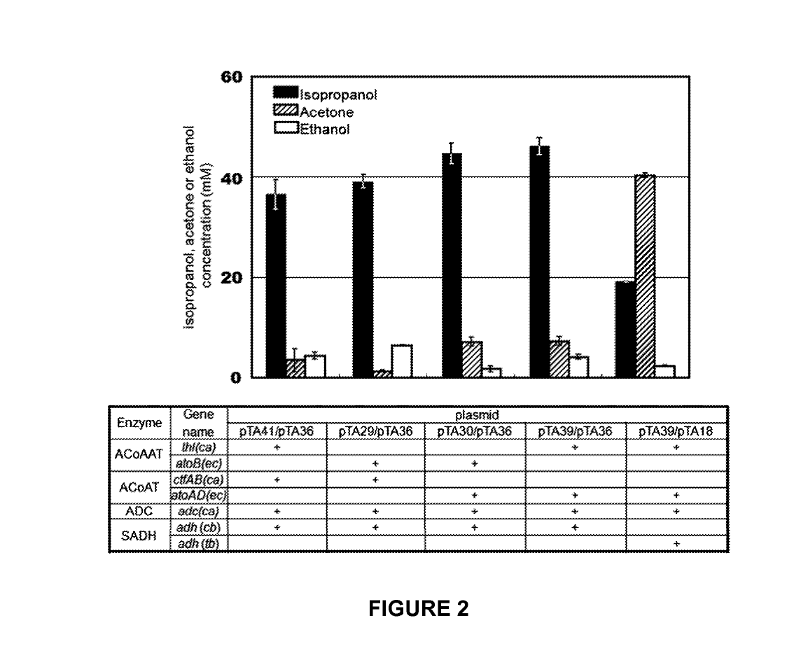

Five different gene combinations for isopropanol production (FIG. 2) were used. All synthetic pathways produced isopropanol from an initial glucose concentration of 111 mM (20 g/l) under aerobic conditions. For all combinations, glucose was exhausted within 12 hours after inoculation (9 hours after induction with IPTG). As shown in FIG. 2, TA11 with pTA39/pTA36 produced the highest amount of isopropanol. The ethanol amounts produced by all strains were very low (less than 10 mM) compared with the isopropanol production.

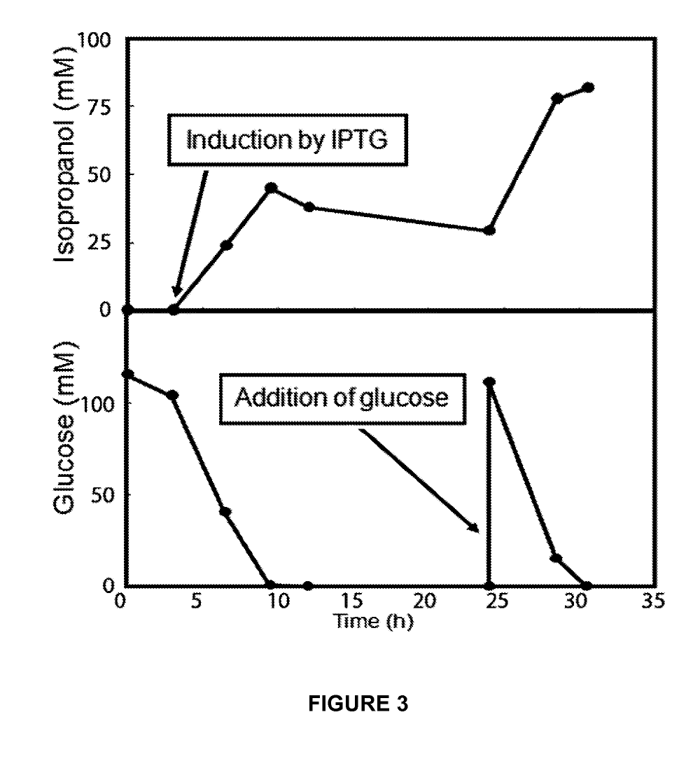

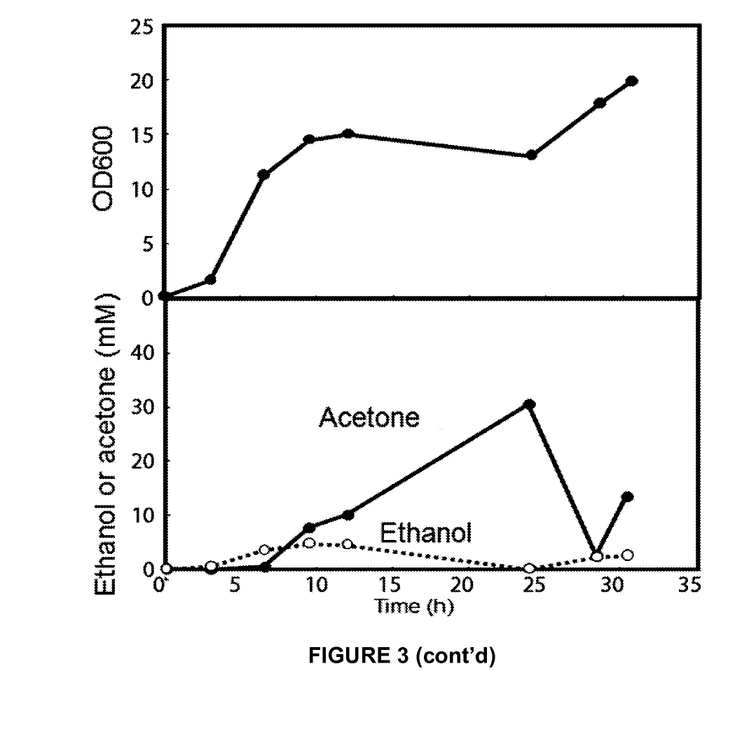

FIG. 3 shows the time course of fermentation using TA11 (pTA39/pTA36). The isopropanol concentration decreased after exhaustion of glucose (12 h). Addition of glucose (111 mM) to the culture at 24 h restored isopropanol production to the same rate as the first production phase, indicating that the pathway activity was stable even after 14 hours of starvation. When glucose was exhausted (30.5 h), the final concentration of isopropanol achieved was 81.6 mM. Acetone concentration continued to increase after exhaustion of initial glucose and suddenly decreased with addition of glucose (FIG. 3). No organic acids except for fumaric acid (maximum concentration, 386 μM) were significantly accumulated after induction by IPTG. The isopropanol concentration decreased while acetone concentration increased during glucose starvation.

Isopropanol production was compared from the strain with pTA36 or pTA18. The amino acid sequence of SADH from C. beijerinckii has a 76% identity and 86% similarity with that from T. brockii. However, TA11 (pTA39/pTA18) produced lower concentrations of isopropanol and much higher concentrations of acetone than the strain containing adh from C. beijerinckii (pTA39/pTA36). To investigate further, the enzyme activity in crude extracts from cultures containing these two alcohol dehydrogenases (either pTA18 or pTA36) was measured. The activity of alcohol dehydrogenase from C. beijerinckii (pTA39/pTA36) (3.08±0.36 unit/mg of protein) was indeed much higher than that from T. brockii (pTA39/pTA18) (0.24±0.08 unit/mg of protein), consistent with the higher isopropanol production.

When the adh-containing plasmid was omitted in the strain, the host produced acetone by TA11 containing only pTA39. At 24 h glucose was added to 111 mM, and the strain continued to produce acetone with almost the same production rate. Cell growth and ethanol concentration displayed a similar tendency as that observed during the isopropanol production experiment. When glucose was exhausted (30.5 h), the final concentration of acetone was measured to be 148.3 mM. As in the isopropanol production, no organic acids except for fumaric acid (maximum concentration: 292 μM) were significantly accumulated after induction of IPTG.

The disclosure demonstrates isopropanol production in E. coli. In shake flask cultures, strain TA11 containing pTA39/pTA36 produced 81.6 mM isopropanol at 30.5 h with a maximum productivity of 6.9 mM/h (0.41 g/l/h) between 3 to 9.5 h. The engineered E. coli surpassed the best reported strain of C. beijerinckii NRRL B593 which produces isopropanol at ˜30 mM with a maximum productivity of ˜3 mM/h (˜0.18 g/l/h) (4). The isopropanol yield at 9.5 h after inoculation was 43.5% (mol isopropanol/mol glucose). The yield is calculated from isopropanol produced (44.8 mM) and the glucose consumed (103.0 mM) between 3 to 9.5 h: molar yield=44.8/103.0=0.435. This yield is very encouraging since the maximum theoretical yield from glucose using the production pathway is 1 (mol isopropanol/mol glucose). The theoretical yield of isopropanol production is calculated based on the pathway shown in FIG. 1. One mole of glucose is converted to 2 moles acetyl-CoA and 2 moles of CO₂. The two acetyl-CoA are then condensed to form one mole of isopropanol, losing one additional mole of CO₂.

The results showed that E. coli containing atoAD produced isopropanol to a higher concentration compared with the strain containing C. beijerinckii ctfAB. As The Km for acetate for CtfAB (1200 mM) is much higher than that of AtoAD (53.1 mM). This difference in acetate affinity likely explains why AtoAD is a better enzyme for isopropanol production. Isopropanol production showed that the conversion rate of SADH from C. beijerinckii from acetone to isopropanol is much higher than that from T. brockii. Indeed, the alcohol dehydrogenase assay using crude extract showed that SADH from C. beijerinckii has a higher activity for isopropanol formation from acetone than that from T. brockii. Although the difference in expression level cannot be ruled out, the SADH from C. beijerinckii is a better choice for isopropanol production under these conditions. Codon optimization may modified expression. It is generally known that the GC content of the gene affects the expression efficiency. The GC content of SADH in C. beijerinckii and in T. brockii is 38 and 44%, respectively. These values are different from the average GC content of E. coli K-12 MG1655.

Bermejo et al., supra, demonstrated batch and glucose fed batch culture using shake flask for acetone production in E. coli, utilizing a recombinant acetone pathway from C. acetobutylicum (thl, ctfAB, adc). This strain produced about 40 mM acetone at 24 h after inoculation in batch culture and 93 mM acetone in a glucose fed batch culture. The same medium was used, the same glucose concentration, and a modified version of the E. coli B strain. Nevertheless, the batch production using same gene construction (pTA41) achieved 60.3 mM of acetone at 12 h. In shake flasks, strain TA11 containing pTA39 achieved a maximum concentration of 148.3 mM at about 30 h, and a maximum productivity of 12.1 mM/h (0.70 g/l/h) (3-9.5 h). The acetone titer also exceeded that produced by wild type C. acetobutylicum (90 mM). Without further optimization of the strain and the fermentation conditions, the acetone yield at 12 h after inoculation was already 73.5% (mol/mol) of the theoretical maximum. This high yield indicates a great potential for using metabolically engineered E. coli in industrial production of isopropanol.

A number of embodiments of the invention have been described. Nevertheless, it will be understood that various modifications may be made without departing from the spirit and scope of the invention. Accordingly, other embodiments are within the scope of the following claims.

1. A recombinant microorganism comprising a biochemical pathway to produce isopropanol from fermentation of a suitable carbon substrate, the biochemical pathway comprising an acetyl-CoA acetyltransferase, wherein the microorganism comprises at least one heterologous polypeptide compared to a corresponding parental microorganism.

2. The recombinant microorganism of claim 1, comprising elevated expression of a polypeptide having acetyl-CoA acetyltransferase activity, as compared to a parental microorganism, wherein the recombinant microorganism produces a metabolite comprising acetoacetyl-CoA from a substrate comprising acetyl-CoA.

3. The recombinant microorganism of claim 2, wherein the polypeptide having acetyl-CoA acetyltranferase activity is encoded by a polynucleotide having at least about 50% identity to a sequence as set forth in SEQ ID NO:30, 66, 68, or 66 and 68.

4. The recombinant microorganism of claim 2, wherein the polypeptide having acetyl-CoA acetyltranferase activity is encoded by an atoB gene or homolog thereof, or a fadA gene or homolog thereof.

5. The recombinant microorganism of claim 4, wherein the atoB gene or fadA gene is derived from the genus Escherichia.

6. The recombinant microorganism of claim 5, wherein the Escherichia is E. coli.

7. The recombinant microorganism of claim 1, wherein the microorganism is a recombinant E. coli.

8. The recombinant microorganism of claim 7, wherein the polypeptide having acetyl-coA acetyltransferase activity is encoded by a polynucleotide having at least about 50% identity to a sequence as set forth in SEQ ID NO:32.

9. The recombinant microorganism of claim 7, wherein the polypeptide having acetyl-CoA acetyltransferase activity is encoded by a thl gene or homolog thereof.

10. The recombinant microorganism of claim 9, wherein the thl gene is derived from the genus Clostridium.

11. The recombinant microorganism of claim 10, wherein the Clostridium is C. acetobutylicum.

12. The recombinant microorganism of claim 1, comprising elevated expression of a polypeptide having acetoacetyl-CoA transferase activity, as compared to a parental microorganism, wherein the recombinant microorganism produces a metabolite comprising acetoacetate from a substrate comprising acetoacetyl-CoA.

13. The recombinant microorganism of claim 12, wherein the acetoacetyl-CoA transferase is encoded by an atoAD gene or homolog thereof.

14. The recombinant microorganism of claim 13, wherein the atoAD gene is derived from E. coli.

15. The recombinant microorganism of claim 1, comprising elevated expression of a polypeptide having acetoacetate decarboxylase activity, as compared to a parental microorganism, wherein the recombinant microorganism produces a metabolite comprising acetone from a substrate comprising acetoacetate.

16. The recombinant microorganism of claim 15, wherein the acetoacetate decarboxylase is encoded by a adc gene or homolog thereof.

17. The recombinant microorganism of claim 16, wherein the adc gene is derived from Clostridium acetobutylicum, Butyrivibrio fibrisolvens, Thermoanaerobacterium thermosaccharolyticum, and Clostridium difficile.

18. The recombinant microorganism of claim 17, wherein the microorganism is Clostridium acetobutylicum.

19. The recombinant microorganism of claim 1, comprising elevated expression of a polypeptide having secondary alcohol dehydrogenase activity, as compared to a parental microorganism, wherein the recombinant microorganism produces a metabolite comprising isopropanol from a substrate comprising acetone.

20. The recombinant microorganism of claim 19, wherein the polypeptide having secondary alcohol dehydrogenase activity is encoded by a polynucleotide having at least about 50% identity to a sequence as set forth in any one of SEQ ID NOs:7 or 9.

21. The recombinant microorganism of claim 19, wherein the polypeptide having secondary alcohol dehydrogenase activity is encoded by a adh or sadh gene or homolog thereof.

22. (canceled)

23. The recombinant microorganism of claim 1, wherein the suitable carbon substrate comprises glucose.

24. The recombinant microorganism of claim 1, wherein the recombinant microorganism comprises one or more deletions or knockouts in a gene encoding an enzyme that catalyzes the conversion of acetyl-coA to ethanol, catalyzes the conversion of pyruvate to lactate, catalyzes the conversion of acetyl-coA and phosphate to coA and acetyl phosphate, catalyzes the conversion of acetyl-coA and formate to coA and pyruvate, or condensation of the acetyl group of acetyl-CoA with 3-methyl-2-oxobutanoate (2-oxoisovalerate).

25. The recombinant microorganism of claim 1, further comprising reduced ethanol dehydrogenase activity, lactate dehydrogenase activity, phosphate acetyltransferase activity, or any combination thereof.

26. The recombinant microorganism of claim 25, wherein the knockout or disruption comprises a deletion or disruption selected from the group consisting of adhE, IdhA, any combination thereof, any homolog or naturally occurring variants thereof.

27. The recombinant microorganism of claim 1, comprising an expression or increased expression of a acetyl-CoA acetyltransferase, an acetoacetyl-CoA transferase, an acetoacetate decarboxylase and a secondary alcohol dehydrogenase.

28. A recombinant microorganism comprising a recombinant biochemical pathway to produce isopropanol from fermentation of a suitable carbon substrate, wherein the recombinant biochemical pathway comprises elevated expression of an acetyl-CoA acetyltransferase, an acetoacetyl-CoA transferase, an acetoacetate decarboxylase and a secondary alcohol dehydrogenase.

29. The recombinant microorganism of claim 28, wherein the suitable carbon substrate comprises glucose.

30. A method of producing a recombinant microorganism that converts a suitable carbon substrate to isopropanol, the method comprising transforming a microorganism with one or more polynucleotides encoding polypeptides having an acetyl-CoA acetyltransferase, an acetoacetyl-CoA transferase, an acetoacetate decarboxylase and a secondary alcohol dehydrogenase activity.

31. The method of claim 30, wherein the suitable carbon substrate comprises glucose.

32. A method for producing isopropanol, the method comprising inducing over-expression of an thl or atoB gene, a ctfAB or atoAD gene or operon, an adc gene, a secondary alcohol dehydrogenase gene, or any combination thereof, in an organism, wherein the organism produces isopropanol when cultured in the presence of a suitable carbon substrate.

33. A method for producing isopropanol, the method comprising:
(i) inducing over-expression of a thl or atoB gene in an organism;
(ii) inducing over-expression of an of a ctfAB or atoAD gene in an organism;
(iii) inducing over-expression of a adc gene in the organism;
(iv) inducing over-expression of an adh gene in the organism; or
(v) inducing over-expression of (i), (ii), (iii), and (iv).

34. The method of claim 32 or claim 33, wherein the suitable carbon substrate comprises glucose.

35. A recombinant vector comprising:
(i) a first polynucleotide encoding a first polypeptide that catalyzes the conversion of acetoacetate from a substrate comprising acetoacetyl-CoA;
(ii) a second polynucleotide encoding a second polypeptide the catalyzes the conversion of acetone from a substrate comprising acetoacetate; and
(iii) a third polynucleotide encoding a third polypeptide that catalyzes the conversion of isopropanol from a substrate comprising acetone.

36. The recombinant vector of claim 35, wherein the first polynucleotide encodes an acetyl-CoA acetyl transferase or an acetoacetyl-CoA transferase.

37. The recombinant vector of claim 35, wherein the second polynucleotide encodes an acetoacetate decarboxylase.

38. The recombinant vector of claim 35, wherein the third polynucleotide encodes a secondary alcohol dehydrogenase.

39. The recombinant vector of claim 35, wherein the vector comprises of C. acetobutylicum thl (acetyl-CoA acetyltransferase), E. coli atoAD (acetoacetyl-CoA transferase), C. acetobutylicum adc (acetoacetate decarboxylase) and C. beijerinckii adh (secondary alcohol dehydrogenase) genes or polynucleotides.

40. The recombinant vector of claim 35, 36, 37, 38 or 39, transfected into an E. coli.

41. The recombinant vector of claim 35, wherein the vector is a plasmid.

42. The recombinant vector of claim 35, wherein the vector is an expression vector.

43. A recombinant host cell comprising the expression vector of claim 42.

44. The recombinant host cell of claim 43, wherein the host cell produced isopropanol.

45. A recombinant microorganism comprising at least one heterologous nucleic acid sequence that facilitates the conversion of glucose to isopropanol.


SEQUENCE LISTING (23 sequences)

SEQ ID NO: 1 - DNA, 1185 bp, Escherichia coli
  CDS (1)..(1185)
atgaaaaattgtgtcatcgtcagtgcggtacgtactgctatcggtagttttaacggttcactcgcttccaccagcgccatcgacctgggggcgacagtaattaaagccgccattgaacgtgcaaaaatcgattcacaacacgttgatgaagtgattatgggtaacgtgttacaagccgggctggggcaaaatccggcgcgtcaggcactgttaaaaagcgggctggcagaaacggtgtgcggattcacggtcaataaagtatgtggttcgggtcttaaaagtgtggcgcttgccgcccaggccattcaggcaggtcaggcgcagagcattgtggcggggggtatggaaaatatgagtttagccccctacttactcgatgcaaaagcacgctctggttatcgtcttggagacggacaggtttatgacgtaatcctgcgcgatggcctgatgtgcgccacccatggttatcatatggggattaccgccgaaaacgtggctaaagagtacggaattacccgtgaaatgcaggatgaactggcgctacattcacagcgtaaagcggcagccgcaattgagtccggtgcttttacagccgaaatcgtcccggtaaatgttgtcactcggaagaaaaccttcgtcttcagtcaagacgaattcccgaaagcgaattcaacggctgaagcgttaggtgcattgcgcccggccttcgataaagcaggaacagtcaccgctgggaacgcgtctggtattaacgacggtgctgccgctctggtgattatggaagaatctgcggcgctggcagcaggccttacccccctggctcgcattaaaagttatgccagcggtggcgtgccccccgcattgatgggtatggggccagtacctgccacgcaaaaagcgttacaactggcggggctgcaactggcggatattgatctcattgaggctaatgaagcatttgctgcacagttccttgccgttgggaaaaccctgggctttgattctgagaaagtgaatgtcaacggcggggccatcgcgctcgggcatcctatcggtgccagtggtgctcgtattctggtcacactattacatgccatgcaggcacgcgataaaacgctggggctggcaacactgtgcattggcggcggccagggaattgcgatggtgattgaacggttgaattaa

SEQ ID NO: 2 - protein, 394 aa, Escherichia coli
MKNCVIVSAVRTAIGSFNGSLASTSAIDLGATVIKAAIERAKIDSQHVDEVIMGNVLQAGLGQNPARQALLKSGLAETVCGFTVNKVCGSGLKSVALAAQAIQAGQAQSIVAGGMENMSLAPYLLDAKARSGYRLGDGQVYDVILRDGLMCATHGYHMGITAENVAKEYGITREMQDELALHSQRKAAAAIESGAFTAEIVPVNVVTRKKTFVFSQDEFPKANSTAEALGALRPAFDKAGTVTAGNASGINDGAAALVIMEESAALAAGLTPLARIKSYASGGVPPALMGMGPVPATQKALQLAGLQLADIDLIEANEAFAAQFLAVGKTLGFDSEKVNVNGGAIALGHPIGASGARILVTLLHAMQARDKTLGLATLCIGGGQGIAMVIERLN

SEQ ID NO: 3 - DNA, 663 bp, Escherichia coli
  CDS (1)..(663)
atgaaaacaaaattgatgacattacaagacgccaccggcttctttcgtgacggcatgaccatcatggtgggcggatttatggggattggcactccatcccgcctggttgaagcattactggaatctggtgttcgcgacctgacattgatagccaatgataccgcgtttgttgataccggcatcggtccgctcatcgtcaatggtcgagtccgcaaagtgattgcttcacatatcggcaccaacccggaaacaggtcggcgcatgatatctggtgagatggacgtcgttctggtgccgcaaggtacgctaatcgagcaaattcgctgtggtggagctggacttggtggttttctcaccccaacgggtgtcggcaccgtcgtagaggaaggcaaacagacactgacactcgacggtaaaacctggctgctcgaacgcccactgcgcgccgacctggcgctaattcgcgctcatcgttgcgacacacttggcaacctgacctatcaacttagcgcccgcaactttaaccccctgatagcccttgcggctgatatcacgctggtagagccagatgaactggtcgaaaccggcgagctgcaacctgaccatattgtcacccctggtgccgttatcgaccacatcatcgtttcacaggagagcaaataa

SEQ ID NO: 4 - protein, 220 aa, Escherichia coli
MKTKLMTLQDATGFFRDGMTIMVGGFMGIGTPSRLVEALLESGVRDLTLIANDTAFVDTGIGPLIVNGRVRKVIASHIGTNPETGRRMISGEMDVVLVPQGTLIEQIRCGGAGLGGFLTPTGVGTVVEEGKQTLTLDGKTWLLERPLRADLALIRAHRCDTLGNLTYQLSARNFNPLIALAADITLVEPDELVETGELQPDHIVTPGAVIDHIIVSQESK

SEQ ID NO: 5 - DNA, 735 bp, Clostridium acetobutylicum
  CDS (1)..(735)
atgttaaaggatgaagtaattaaacaaattagcacgccattaacttcgcctgcatttcctagaggaccctataaatttcataatcgtgagtattttaacattgtatatcgtacagatatggatgcacttcgtaaagttgtgccagagcctttagaaattgatgagcccttagtcaggtttgaaattatggcaatgcatgatacgagtggacttggttgttatacagaaagcggacaggctattcccgtaagctttaatggagttaagggagattatcttcatatgatgtatttagataatgagcctgcaattgcagtaggaagggaattaagtgcatatcctaaaaagctcgggtatccaaagctttttgtggattcagatactttagtaggaactttagactatggaaaacttagagttgcgacagctacaatggggtacaaacataaagccttagatgctaatgaagcaaaggatcaaatttgtcgccctaattatatgttgaaaataatacccaattatgatggaagccctagaatatgtgagcttataaatgcgaaaatcacagatgttaccgtacatgaagcttggacaggaccaactcgactgcagttatttgatcacgctatggcgccacttaatgatttgccagtaaaagagattgtttctagctctcacattcttgcagatataatattgcctagagctgaagttatatatgattatcttaagtaa

SEQ ID NO: 6 - protein, 244 aa, Clostridium acetobutylicum
MLKDEVIKQISTPLTSPAFPRGPYKFHNREYFNIVYRTDMDALRKVVPEPLEIDEPLVRFEIMAMHDTSGLGCYTESGQAIPVSFNGVKGDYLHMMYLDNEPAIAVGRELSAYPKKLGYPKLFVDSDTLVGTLDYGKLRVATATMGYKHKALDANEAKDQICRPNYMLKIIPNYDGSPRICELINAKITDVTVHEAWTGPTRLQLFDHAMAPLNDLPVKEIVSSSHILADIILPRAEVIYDYLK

SEQ ID NO: 7 - DNA, 1059 bp, Thermoanaerobacter brockii
  CDS (1)..(1059)
atgaaaggttttgcaatgctgtccatcgggaaagtcggctggattgaaaaagagaaacctgcgccaggtccattcgatgcaatcgttcgcccgctggcggttgcgccttgcacttcagatatccataccgtgtttgaaggcgctatcggcgagcgtcacaacatgattctgggccacgaagccgtgggcgaagtagtagaagtcggctctgaagtgaaggattttaagcctggtgatcgggttgtggtgccggcaattacgccggattggcggacctctgaggtgcaacgtggctatcatcagcatagcggtggtatgctggctggctggaaatttagcaacgtcaaggatggtgtgtttggcgagttttttcatgtcaatgacgcggacatgaatctggcacacctgccgaaagaaattccgttagaagcggccgttatgattcctgacatgatgactacgggctttcatggcgcggaattagcggacattgaactgggcgctacggttgccgttctgggtatcggtccggtgggcctgatggctgtagccggtgccaagctgcgtggtgcgggtcgcatcattgcggtaggttcccgtccagtttgtgttgatgccgcgaaatactatggtgctaccgacatcgtaaactataaagacggtccgattgaaagtcagattatgaacctgacagaaggcaaaggcgttgatgccgcaatcattgccggcggcaatgcagacatcatggctacagccgtaaagatcgtcaaaccaggcgggaccatcgcaaacgtcaattacttcggggagggtgaagtgctgccggtgccgcgtctggaatgggggtgtggtatggcgcacaagaccatcaaagggggtctgtgcccgggggggcgtctgcgcatggagcgcctgattgatctggtcttctataaacgcgttgatccgagtaaattagttacccatgtgttccgcgggttcgataatattgagaaagctttcatgttaatgaaagataaaccgaaagatttaattaaaccagtggtgattttagcatag

SEQ ID NO: 8 - protein, 352 aa, Thermoanaerobacter brockii
MKGFAMLSIGKVGWIEKEKPAPGPFDAIVRPLAVAPCTSDIHTVFEGAIGERHNMILGHEAVGEVVEVGSEVKDFKPGDRVVVPAITPDWRTSEVQRGYHQHSGGMLAGWKFSNVKDGVFGEFFHVNDADMNLAHLPKEIPLEAAVMIPDMMTTGFHGAELADIELGATVAVLGIGPVGLMAVAGAKLRGAGRIIAVGSRPVCVDAAKYYGATDIVNYKDGPIESQIMNLTEGKGVDAAIIAGGNADIMATAVKIVKPGGTIANVNYFGEGEVLPVPRLEWGCGMAHKTIKGGLCPGGRLRMERLIDLVFYKRVDPSKLVTHVFRGFDNIEKAFMLMKDKPKDLIKPVVILA

SEQ ID NO: 9 - DNA, 1056 bp, Clostridium beijerinckii
  CDS (1)..(1056)
atgaaagggtttgccatgttaggtatcaataaactgggctggattgaaaaagagcgcccggtggcgggttcatacgatgcaattgttcgtccgctggccgtcagtccgtgcaccagcgacatccatacagtcttcgaaggtgcactgggtgatcggaaaaacatgattctgggccatgaagccgtaggcgaagtagtggaagtgggcagcgaggtaaaggatttcaaaccgggcgatcgcgtaattgttccttgcacgaccccagattggcgctcactggaagttcaggctggttttcagcagcatagtaacggtatgttagcaggctggaagtttagcaattttaaagacggggtgttcggggagtattttcatgtcaacgatgcggacatgaatctggctattttacctaaagatatgccgctggagaacgcagtgatgattaccgacatgatgacgacaggctttcacggtgccgaactggctgacatccaaatgggctccagtgtggtggttatcggtattggtgcggtcgggctgatgggtatcgcgggcgcgaaattacggggcgctggtcgcatcatcggtgtcggcagccgtccaatttgcgttgaagcagctaaattctatggtgccacggacattctgaactataaaaatggtcacatcgtcgatcaggtgatgaaactgaccaatggcaaaggtgtggaccgcgtgatcatggcgggcggcggctcagagactttatctcaagcggtgtctatggttaaacctgggggcatcatttctaatattaactatcatggctccggcgacgcattactgatcccgcgtgttgaatggggctgtgggatggcccacaaaaccattaaaggggggttatgtccgggtggtcgcctgcgtgccgaaatgctgcgtgacatggtggtttacaaccgtgtggatctgtccaaactggtaactcacgtataccacggtttcgatcacattgaagaggcgctgctgctgatgaaggataagccaaaggatctgattaaggcggttgttatcctgtaa

SEQ ID NO: 10 - protein, 351 aa, Clostridium beijerinckii
MKGFAMLGINKLGWIEKERPVAGSYDAIVRPLAVSPCTSDIHTVFEGALGDRKNMILGHEAVGEVVEVGSEVKDFKPGDRVIVPCTTPDWRSLEVQAGFQQHSNGMLAGWKFSNFKDGVFGEYFHVNDADMNLAILPKDMPLENAVMITDMMTTGFHGAELADIQMGSSVVVIGIGAVGLMGIAGAKLRGAGRIIGVGSRPICVEAAKFYGATDILNYKNGHIVDQVMKLTNGKGVDRVIMAGGGSETLSQAVSMVKPGGIISNINYHGSGDALLIPRVEWGCGMAHKTIKGGLCPGGRLRAEMLRDMVVYNRVDLSKLVTHVYHGFDHIEEALLLMKDKPKDLIKAVVIL

SEQ ID NO: 11 - DNA, 15 bp, Artificial sequence
  Ribosome binding site
gaaggagatatacat

SEQ ID NO: 12 - DNA, 34 bp, Artificial Sequence
  Oligonucleotide primer
cgcggtaccatgaaaaattgtgtcatcgtcagtg

SEQ ID NO: 13 - DNA, 35 bp, Artificial Sequence
  Oligonucleotide primer
ccgcgtcgacttaattcaaccgttcaatcaccatc

SEQ ID NO: 14 - DNA, 50 bp, Artificial Sequence
  Oligonucleotide primer
ccgcgtcgacgaaggagatatacatatgaactctaaaataattagatttg

SEQ ID NO: 15 - DNA, 34 bp, Artificial Sequence
  Oligonucleotide primer
cgccccgggctaaacagccatgggtctaagttca

SEQ ID NO: 16 - DNA, 49 bp, Artificial Sequence
  Oligonucleotide primer
cgccccggggaaggagatatacatatgttaaaggatgaagtaattaaac

SEQ ID NO: 17 - DNA, 34 bp, Artificial Sequence
  Oligonucleotide primer
cgcggatccttacttaagataatcatatataact

SEQ ID NO: 18 - DNA, 50 bp, Artificial Sequence
  Oligonucleotide primer
ccgcgtcgacgaaggagatatacatatgaaaacaaaattgatgacattac

SEQ ID NO: 19 - DNA, 34 bp, Artificial Sequence
  Oligonucleotide primer
cgccccgggtcataaatcaccccgttgcgtattc

SEQ ID NO: 20 - DNA, 34 bp, Artificial Sequence
  Oligonucleotide primer
cgcggtaccatgaaagaagttgtaatagctagtg

SEQ ID NO: 21 - DNA, 34 bp, Artificial Sequence
  Oligonucleotide primer
ccgcgtcgacctagcacttttctagcaatattgc

SEQ ID NO: 22 - DNA, 34 bp, Artificial Sequence
  Oligonucleotide primer
cgcggtaccatgaaaggttttgcaatgctgtcca

SEQ ID NO: 23 - DNA, 37 bp, Artificial Sequence
  Oligonucleotide primer
cgctctagactatgctaaaatcaccactggtttaatt